(19) United States
(12) Patent Application Publication
(10) Pub. No.: US 2004/0116348 A1
(43) Pub. Date: Jun. 17, 2004

(54) Polymer-linker-drug conjugates for targeted drug delivery

(76) Inventors: Ying Chau, Cambridge, MA (US); Robert S. Langer, Newton, MA (US)

Correspondence Address: Choate, Hall & Stewart, Exchange Place, 53 State Street, Boston, MA, 02109, US

(21) Appl. No.: 10/668,045

(22) Filed: Sep. 22, 2003

Related U.S. Application Data
(60) Provisional application No. 60/412,760, filed on Sep. 23, 2002.

(51) Int. Cl. A61K 38/00

(52) U.S. Cl. 514/12

(57) ABSTRACT

A system for selectively delivering drugs to target tissues is provided. The system includes a polymer-linker-drug conjugate. The linker includes a segment that is recognized and cleaved by a digestive enzyme that is overexpressed in the extracellular space of the target tissue. The recognition segment is preferably an oligopeptide or oligosaccharide segment. The polymeric carrier is preferably hydrophilic, biodegradable and biocompatible particle. Any drug may be delivered using a conjugate prepared according to the invention. Methods of preparing the conjugates and administering the conjugates for targeted drug delivery are also provided.

### RELATED APPLICATIONS

The present application claims priority to provisional application U.S. Serial No. 60/412,760, filed Sep. 23, 2002 which is incorporated herein by reference in its entirety.

### BACKGROUND OF THE INVENTION

Despite numerous recent advances in drug delivery technology (Langer, Nature 392:5, 1998 and Langer, Science 293:58, 2001) there remains a need in the art for methods, devices and compositions for controllably and preferentially delivering drugs to diseased tissues. The ability to control the precise level and location of drugs in a patient would allow doses to be reduced, minimize side effects and open new avenues for drug therapy. Targeted drug delivery systems are particularly desirable in the treatment of tissue specific diseases such as cancer that are currently treated by systemic chemotherapy.

### SUMMARY OF THE INVENTION

The present invention provides a system for selectively delivering drugs to target tissues. In preferred embodiments, the target tissues are diseased tissues. The inventive delivery system includes a polymer-linker-drug conjugate. The linker includes a segment that is recognized and cleaved by a digestive enzyme that is overexpressed in the extracellular space of the target tissue. The recognition segment is preferably an oligopeptide or oligosaccharide segment. The linker may also include a spacer that separates the recognition segment from the polymeric carrier and/or drug. Without wishing to be bound to any particular theory, when the conjugate reaches the target tissue the recognition segment within the linker is thought to be cleaved by the digestive enzyme. The active drug is thereby released from the conjugate and subsequently internalized by the cells of the target tissue. The polymeric carrier is preferably hydrophilic, biodegradable and biocompatible. In preferred embodiments the polymeric carrier is greater in size than the renal excretion limit. The physiochemical features of the polymeric carrier allow the conjugate to circulate longer in plasma by decreasing renal excretion and liver clearance. The polymeric carrier may be loaded with any number of drug molecules. In particular it is to be understood that the conjugate may include a single drug molecule or a plurality of drug molecules each attached to the polymeric carrier via an inventive linker. It is also to be understood that any drug molecule whether a small molecule drug or a biomolecular drug (e.g., a therapeutic protein or nucleic acid) may be delivered using a conjugate prepared according to the invention.

The present invention also provides methods of administering the inventive conjugates and pharmaceutical compositions comprising the inventive conjugates to a patient in need thereof. The inventive conjugates and/or compositions once prepared can be administered to the patient by any means known in the art.

### Definitions

“Digestive enzyme”: As used herein, a “digestive enzyme” is an enzyme that cleaves polymers. Preferably the cleaved polymers are oligopeptides or oligosaccharides. Digestive enzymes of the present invention exhibit some form of specificity. For example, a digestive enzyme that cleaves oligopeptides will typically exhibit strong selectivity for oligopeptides that include one or a small subset of amino acid sequences called recognition sequences. As is further described herein and as is known in the art, the specificity and recognition sequence of a particular digestive enzyme may be determined by comparing the rate at which it cleaves different polymers within a given family (e.g., oligopeptides or oligosaccharides having different sequences).

“Oligopeptide”: According to the present invention, an “oligopeptide” comprises a string of at least three amino acid residues linked together by peptide bonds. The terms “oligopeptide”, “peptide”, “polypeptide” and “protein” may be used interchangeably. Inventive oligopeptides preferably contain only natural amino acid residues, although non-natural amino acids (i.e., compounds that do not occur in nature but that can be incorporated into a polypeptide chain) and/or amino acid analogs as are known in the art may alternatively be employed. Also, one or more of the amino acid residues in an inventive oligopeptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the oligopeptide lead to a more stable oligopeptide (e.g., greater stability to digestion by enzymes in the gastrointestinal tract). These modifications may include cyclization of the peptide, the incorporation of D-amino acids, etc. If the modifications are made to an oligopeptide drug molecule, they should not substantially interfere with the desired biological activity of the oligopeptide.

“Oligonucleotide”: According to the present invention, an “oligonucleotide” comprises a string of at least three nucleotides linked together by phosphodiester bonds. The terms “oligonucleotide”, “polynucleotide” and “nucleic acid” may be used interchangeably. Typically, an oligonucleotide comprises at least three nucleosides. Oligonucleotides may include natural nucleosides (i.e., adenosine, thymidine, guanosine, cytidine, uridine, deoxyadenosine, deoxythymidine, deoxyguanosine and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine and 2-thiocytidine), chemically modified bases, biologically modified bases (e.g., methylated bases), intercalated bases, modified sugars (e.g., 2′-fluororibose, ribose, 2′-deoxyribose, arabinose and hexose), or modified phosphate groups (e.g., phosphorothioates and 5′-N-phosphoramidite linkages).

“Oligosaccharide”: The term “oligosaccharide” refers to a polymer of sugars. The terms “oligosaccharide”, “polysaccharide” and “carbohydrate” may be used interchangeably. Typically, an oligosaccharide comprises at least three sugars. The polymer may include natural sugars (e.g., glucose, fructose, galactose, mannose, arabinose, ribose and xylose) and/or modified sugars (e.g., 2′-fluororibose, 2′-deoxyribose and hexose).

“Overexpressed”: As used herein, a digestive enzyme is “overexpressed” in a tissue sample if it is present in excess relative to the abundance of that enzyme in at least one other tissue sample (e.g., when comparing a diseased tissue with a healthy tissue from the same individual). As is well known in the art, there is generally a correlation between overexpression at the RNA level and overexpression at the protein level. In other words, if a mRNA is overexpressed then it is highly likely that the corresponding polypeptide is also overexpressed. Therefore, detection of either mRNA or a corresponding polypeptide is generally sufficient to determine whether a particular polypeptide is overexpressed. However, as is well known in the art, in certain situations it may be more convenient and/or practical to detect mRNA while in other situations it may be more convenient and/or practical to detect polypeptides.

### BRIEF DESCRIPTION OF THE DRAWING

FIG. 1 is a schematic that illustrates the overall structure of an embodiment of an inventive conjugate.

FIG. 2 is a schematic that illustrates how substrate phage display methodology can be used to select suitable oligopeptide sequences for use in linkers of inventive conjugates.

FIG. 3 is a schematic that illustrates the chemical structure of the free drug doxorubicin.

FIG. 4 is a schematic that illustrates the chemical structure of an inventive CM-dextran-oligopeptide-doxorubicin conjugate.

FIGS. 5A-B are graphs that compare the digestion of four different CM-dextran-oligopeptide-doxorubicin conjugates by pure matrix-metalloproteinase It (MMP-2) at 37 C. The conjugates each include a linker with an oligopeptide in the series IPVGLIG (diamond), IPVGLI (cross), IPVGL (square) and IPVG (triangle). The graphs show the concentration of released peptidyl-doxorubicin as a function of digestion time. The data in FIG. 5A were obtained with a doxorubicin-equivalent concentration of 29.5 μM and an MMP-2 concentration of 0.74 μM. The data in FIG. 5B were obtained with a doxorubicin-equivalent concentration of 3.2 μM and an MMP-2 concentration of 100 nM.

FIGS. 6A-F are graphs that compare the cytotoxicity of free doxorubicin (empty squares) and the cleaved peptidyl-doxorubicin (i.e., LIG-doxorubicin, filled squares) on six different human tumor cell lines, namely HT-1080, BT-20, U-87, PC-3, KK-47 and MGH-U1. The graphs show the % survival of tumor cells as a function of the concentration of the doxorubicin-equivalent concentration in the cell culture.

FIG. 7 is a schematic that illustrates the chemical structure of an inventive dextran-poly(ethyleneglycol)-oligopeptide-doxorubicin conjugate.

FIG. 8 is a schematic that illustrates the chemical structure of an inventive methoxy-poly(ethyleneglycol)-oligopeptide-doxorubicin conjugate

FIG. 9 is a schematic that illustrates the chemical structure of the free drug methotrexate.

FIG. 10 is a graph that compares the cytotoxicity of methotrexate-PVG (empty squares), methotrexate-IPVG (empty triangles) and free methotrexate (empty circles) on the human tumor cell line HT-1080. The graphs show the % survival of tumor cells as a function of the concentration of the methotrexate-equivalent concentration in the cell culture.

FIGS. 11A-C are graphs that compare the cytotoxicity of free methotrexate (empty circles) and the cleaved peptidyl-methotrexate (i.e., PVG-methotrexate, filled circles) on three different human tumor cell lines, namely HT-1080, BT-20 and RT-112. The graphs show the % survival of tumor cells as a function of the concentration of the methotrexate-equivalent concentration in the cell culture.

FIGS. 12A-C depict a synthetic scheme for preparing inventive dextran-oligopeptide-methotrexate conjugates of the invention.

FIG. 13 is a graph comparing the tumor progression after different groups of SCID mice bearing HT-1080 tumors were treated with free methotrexate, a dextran-methotrexate conjugate, a dextran-oligopeptide-methotrexate conjugate, modified dextran or phosphate buffered saline (PBS).

FIG. 14 is a graph comparing the body weight change after different groups of SCID mice bearing HT-1080 tumors were treated with free methotrexate, a dextran-methotrexate conjugate, a dextran-oligopeptide-methotrexate conjugate, modified dextran or PBS.

FIG. 15 is a graph comparing the tumor progression after two different groups of SCID mice bearing HT-1080 tumors were treated with two dextran-oligopeptide-methotrexate conjugates having different backbone negative charges.

### DETAILED DESCRIPTION OF CERTAIN PREFERRED EMBODIMENTS OF THE INVENTION

The present application mentions various patents, published patent applications, scientific articles and other publications. The contents of each such item are hereby incorporated by reference.

The present invention provides a system for selectively delivering drugs to target tissues. In preferred embodiments, the target tissues are diseased tissues. The inventive delivery system includes a polymer-linker-drug conjugate. The linker includes a segment that is recognized and cleaved by a digestive enzyme that is overexpressed in the extracellular space of the target tissue. The recognition segment is preferably an oligopeptide or oligosaccharide segment. The linker may also include a spacer that separates the recognition segment from the polymeric carrier and/or drug. Without wishing to be bound to any particular theory, when the conjugate reaches the target tissue the recognition segment within the linker is thought to be cleaved by the digestive enzyme. The active drug is thereby released from the conjugate and subsequently internalized by the cells of the target tissue. The polymeric carrier is preferably hydrophilic, biodegradable and biocompatible. In preferred embodiments the polymeric carrier is greater in size than the renal excretion limit. The physiochemical features of the polymeric carrier allow the conjugate to circulate longer in plasma by decreasing renal excretion and liver clearance. The polymeric carrier may be loaded with any number of drug molecules. In particular it is to be understood that the conjugate may include a single drug molecule or a plurality of drug molecules each attached to the polymeric carrier via an inventive linker. It is also to be understood that any drug molecule whether a small molecule drug or a biomolecular drug (e.g., a therapeutic protein or nucleic acid) may be delivered using a conjugate prepared according to the invention. The polymeric, linker and drug components of the inventive conjugates are described in greater detail below.

Polymeric Carrier

In certain embodiments, the polymeric carrier allows conjugates and hence drugs to circulate longer in plasma by decreasing renal excretion and liver clearance. Strategies for minimizing body clearance have focused on the liver and kidney because they are known to be the major elimination sites in the body. In this context, Hashida and Takakura have reviewed the current status of macromolecular drug delivery systems (Hashida and Takakura, Journal of Controlled Release 31:163, 1994). They related the physiological features of the liver and kidney to the clearance data obtained with macromolecules.

The glomerular capillaries in the kidney are fenestrated, with pores having radii estimated at 20-30 nm. Their basement membranes act as a size and charge barrier which appear to hinder the transport of particles above 6 nm. Consistent with these features, macromolecules with sizes above about 6 nm (MW˜50,000 Da) exhibit marked inhibition on renal clearance. Accordingly, in certain embodiments the polymeric carrier may be designed to be greater than this renal excretion limit. It is to be understood however that the inventive conjugates may also include polymer carriers that are smaller than the renal excretion limit.

In the liver, the basement membrane is absent and capillaries are characterized by fenestrae of about 100 nm and endothelial gaps from 100-1000 nm. The passage of macromolecules is therefore relatively unrestricted. However, cellular uptake of these carriers can be lowered by decreasing interactions between cells and carriers. In general, cationic molecules tend to bind to cell surfaces and strongly anionic species facilitate receptor mediated endocytosis. A slightly anionic carrier, for example, carboxymethyl-dextran (CM-dextran), appears to elicit least uptake by liver cells and its hepatic clearance is similar to that of fluid phase endocytosis (i.e., ˜10 μl/hr in mice).

One additional advantage of using macromolecules as drug carriers in the case of tumor targeting comes from the abnormality of tumor vasculature (Jain, Cancer and Metastasis Reviews 6:559, 1987). Most capillaries are continuous, with small pore radii of 6.7-8.0 nm and large pore radii of 20-28 nm. They are found in muscle, lung, skin, subcutaneous tissues, serous and mucous membranes. Transport of macromolecules above ˜7 nm across these capillaries is negligible. In contrast, tumor blood vessels are leaky and the basement membrane is absent. By observing the tranvascular transport of liposomes under the dorsal skin chamber in tumor-bearing mice, Yuan et al. determined the cutoff pore size of tumor vessels to be 400-600 nm (Yuan et al., Cancer Research 55:3752, 1995). These features are attributed to the expression of angiogenic factors and various collagen degrading enzymes (Maeda et al., Journal of Controlled Release 65:271, 2000). Larger drug carriers can therefore selectively reach malignant tissues while sparing most normal tissues. The liver, bone marrow, spleen and kidney are the key exceptions.

Maeda and co-workers have further observed that macromolecules are also preferentially retained in tumor tissues. They conjugated the anti-tumor protein neocarzinostatin to styrene and maleic acid copolymer (SMANCS) and reported poor recovery of the drug carriers in the effluent blood and lymph. The explanation for this phenomenon, termed enhanced permeability and retention (EPR), is not entirely clear. The researchers attribute this effect to the augmented leakiness of tumor blood vessels and the lack of lymphatic drainage in tumor tissues (Matsumura and Maeda, Cancer Research 46:6387, 1986). Essentially, while the molecules can easily extravasate into the tumor tissue, return to the circulation is blocked.

In addition to controlling the size and charge of drug carrier, Kilbanov and a number of laboratories have demonstrated the benefit of using poly(ethylene-glycol) (PEG) stealth in polymeric micelles (Yokoyama et al., Cancer Research 51:3229, 1991); nanoparticles (Gref et al., Science 263:1600, 1994; Gref et al., Advanced Drug Delivery Reviews 16:215, 1995); and liposomes (Klibanov et al., FEBS Lett. 268:235, 1990). A layer of linear hydrophilic polymers surrounding the carrier in the core provides a shielding effect by avoiding absorption onto the cell surface in the eliminating organ. With the appropriate molecular weight and density, the PEG brush also inhibits proteins from interacting with the core through steric hindrance (VertutDoi et al., Biochimica BiophysicaActa—Biomembranes 1278:19, 1996; Grefet al., Colloids Surfaces B—Biointerfaces 18:301, 2000). These features prolong the circulation of polymeric carriers in the circulation.

In certain preferred embodiments, the polymeric carriers used in an inventive conjugate are biocompatible. Biocompatible polymers are not significantly toxic to cells. In order to prevent chromic accumulation of polymeric carriers that are larger than the renal excretion limit are preferably both biocompatible and biodegradable. Biodegradable polymers are broken down by the cellular machinery and/or by hydrolysis into components that the cells can either reuse or dispose of without significant toxic effect. In preferred embodiments, a biodegradable polymer and its biodegradation byproducts are biocompatible. It is to be understood that any known biodegradable polymer may be incorporated in a conjugate of the present invention. Preferred polymeric carriers are hydrophilic, e.g., they may include polar groups, such as hydroxyl or amine groups; anionic groups, such as carboxylate, sulfonate, sulphate, phosphate, or nitrate groups; or cationic groups, such as protonated amine, quaternary ammonium, or phosphonium groups. Suitable hydrolytically degradable polymers known in the art include for example, certain polyesters, polyanhydrides, polyorthoesters, polyphosphazenes and polyphosphoesters. Other biodegradable polymers known in the art, include, for example, certain carbohydrates, polyhydroxyacids, polypropylfumerates, polycaprolactones, polyamides, poly(amino acids), polyacetals, polyethers, biodegradable polycyanoacrylates and biodegradable polyurethanes. For example, specific biodegradable polymers that may be used in the present invention include but are not limited to alginate, carboxymethyl-alginate, cellulose, polylysine, poly(lactic acid), poly(glycolic acid), poly(caprolactone), poly(lactide-co-glycolide), poly(lactide-co-caprolactone) and poly(glycolide-co-caprolactone). Those skilled in the art will recognize that this is an exemplary, not comprehensive, list of biodegradable polymers. It is further to be understood that inventive conjugates may comprise block co-polymers, graft co-polymers, or adducts of these and other polymers.

The properties of these and other polymers and methods for preparing them are well known in the art. See, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. No. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. Nos. 4,806,621; 4,638,045 to Kohn; and U.S. Pat. No. 4,946,929 to d'Amore; see also Wang et al., J. Am. Chem. Soc. 123:9480, 2001; Lim et al., J. Am. Chem. Soc. 123:2460, 2001; Langer, Acc. Chem. Res. 33:94, 2000; Langer, J. Control. Release 62:7, 1999; and Uhrich et al., Chem. Rev. 99:3181, 1999. More generally, a variety of methods for synthesizing suitable polymers are described in Concise Encyclopedia of Polymer Science and Polymeric Amines and Ammonium Salts, Ed. by Goethals, Pergamon Press, 1980; Principles of polymerization by Odian, John Wiley & Sons, 1991; and Contemporary Polymer Chemistry by Allcock et al., Prentice-Hall, 1981.

Drug

Any drug whether a small molecule drug or a biomolecular drug (e.g., a therapeutic protein, nucleic acid or carbohydrate) may be delivered using a conjugate prepared according to the invention. As used herein, the term “small molecule drug” refers to a molecule, whether naturally-occurring or artificially created (e.g., via chemical synthesis) that has a relatively low molecular weight and that is not a protein, a nucleic acid, or a carbohydrate. Typically, though not necessarily, small molecule drugs are monomeric and have a molecular weight of less than about 1500 g/mol. Preferably, though not necessarily, the drug is one that has already been deemed safe and effective for use by the appropriate governmental agency or body. For example, drugs for human use listed by the FDA under 21 C.F.R. §§ 330.5, 331 through 361 and 440 through 460; drugs for veterinary use listed by the FDA under 21 C.F.R. §§ 500 through 589, incorporated herein by reference, are all considered acceptable for use in inventive conjugates.

Classes of small molecule drugs that can be used in the practice of the present invention include, but are not limited to, anti-AIDS drugs, anti-cancer drugs, antibiotics, immunosuppressants, anti-viral drugs, enzyme inhibitors, neurotoxins, opioids, hypnotics, anti-inflammatory drugs, anti-histamines, lubricants, tranquilizers, anti-convulsants, muscle relaxants and anti-Parkinson drugs, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic drugs, anti-emetics and imaging agents (e.g., for use in computed X-ray tomography, magnetic resonance imaging, positron emission tomography, single photon emission computed tomography, etc.).

A more complete listing of classes and specific drugs suitable for use in the present invention may be found in Pharmaceutical Drugs: Syntheses, Patents, Applications by Axel Kleemann and Jurgen Engel, Thieme Medical Publishing, 1999 and the Merck Index: An Encyclopedia of Chemicals, Drugs and Biologicals, Ed. by Budavari et al., CRC Press, 1996, both of which are incorporated herein by reference.

As used herein, the term “biomolecular drug” refers to a molecule, whether naturally-occurring or artificially created (e.g., via synthetic or recombinant methods) that has a relatively high molecular weight and that is a protein, a nucleic acid or a carbohydrate. Typically, though not necessarily, biomolecular drugs have a molecular weight of more than about 1500 g/mol. Classes of biomolecular drugs that can be used in the practice of the present invention include, but are not limited to, therapeutic proteins (e.g., enzymes, neurotransmitters, hormones, cytokines, cell response modifiers such as growth factors and chemotactic factors, antibodies, haptens, toxins, interferons, etc.) and therapeutic nucleic acids (e.g., aptarmers, ribozymes, anti-sense agents, gene vectors, etc.).

Linker

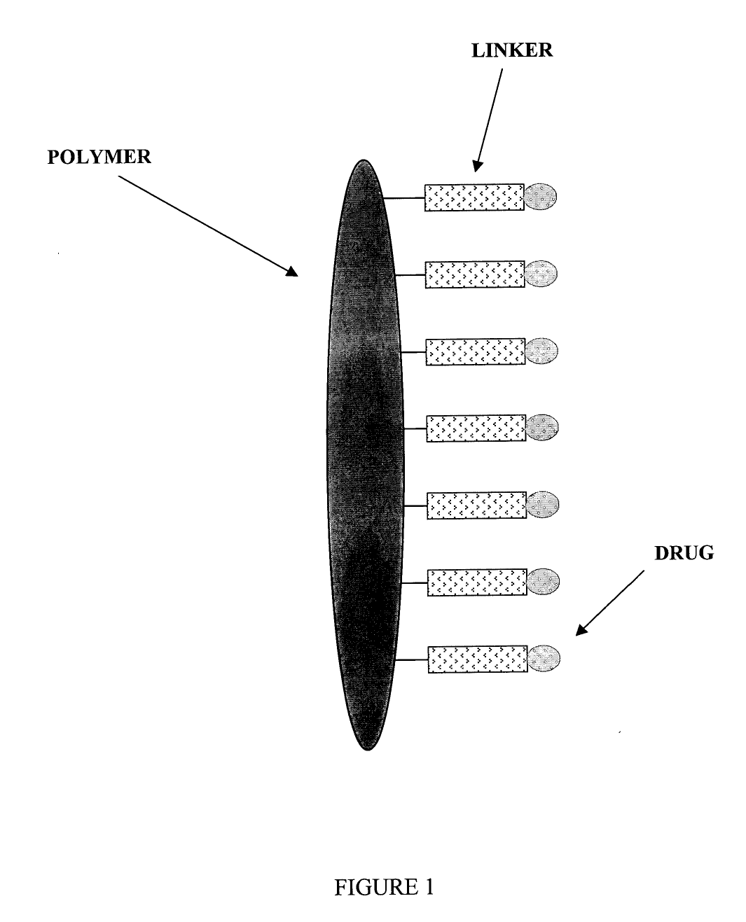

As illustrated in FIG. 1, the conjugates include a linker with at least a first and a second end. The linker includes at least one segment that is cleaved when exposed to a digestive enzyme that is expressed in the target tissue. Preferably the digestive enzyme is overexpressed in the target tissue. In preferred embodiments the cleavable segment includes an oligopeptide or an oligosaccharide sequence. The first end of the linker is associated with the polymeric carrier and the second end is associated with the drug molecule. When two entities are “associated with” one another as described herein, they are linked by a covalent or ligand/receptor type interaction. Preferably, the association is covalent. One of ordinary skill in the art will appreciate that a whole host of synthetic methods exist for covalently linking oligopeptide or oligosaccharide segments with the polymeric carriers and drugs of the present invention. Without limitation, the Examples presented below describe exemplary synthetic methods in greater detail. It is also to be understood that any ligand/receptor pair with a sufficient stability and specificity to operate in the context of the inventive system may be employed to associate two entities. In particular, the ligand/receptor interaction should be sufficiently stable to prevent premature release of the drug molecule from an inventive conjugate (i.e., prior to enzymatic cleavage of the linker). To give but an example, a drug (or polymer) may be covalently linked with biotin and the linker with avidin. The strong binding of biotin to avidin would then allow for association of the drug (or polymer) and linker. In general possible ligand/receptor pairs include antibody/antigen, protein/co-factor and enzyme/substrate pairs. Besides biotin/avidin, these include for example, biotin/streptavidin, FK506/FK506-binding protein (FKBP), rapamycin/FKBP, cyclophilin/cyclosporin and glutathione/glutathione transferase pairs. Other suitable ligand/receptor pairs would be recognized by those skilled in the art.

The chemical composition of the cleavable segment (e.g., the length and sequence of amino acids or sugars) will depend for the most part on the motif that is recognized and cleaved by the digestive enzyme in the target tissue. The cleavage motifs are known for a number of proteases. For example, the table provided in Appendix A lists the cleavage motifs for a range of secreted or membrane bound proteases that are overexpressed in certain tumor tissues. These are all potential target enzymes that could be used to trigger release of drug molecules from inventive conjugates. The Examples describe the optimization of a cleavage segment that is recognized by matrix-metalloproteinase II (MMP-2), a proteinase that is also overexpressed in a variety of tumors.

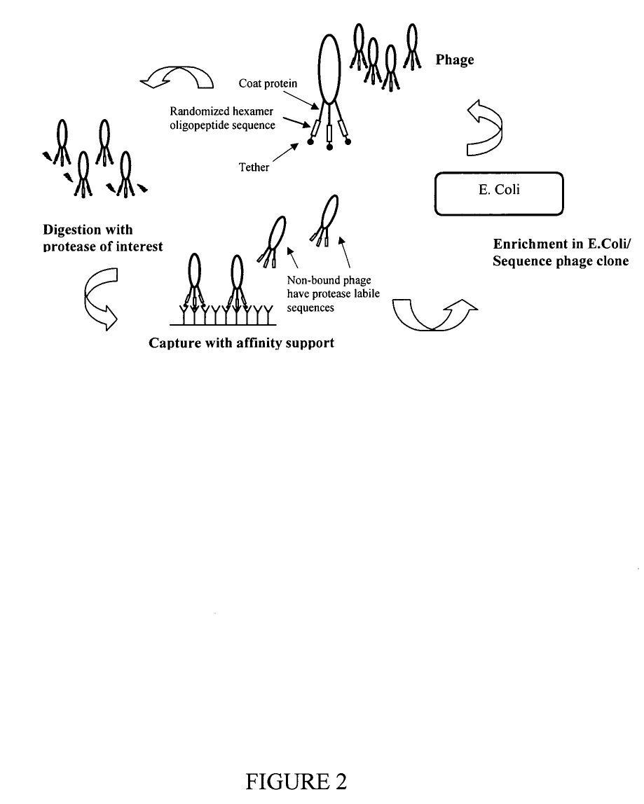

A variety of methods are also known in the art that can be used to determine the cleavage motif of a target enzyme when it is not yet known. These include substrate phage display libraries (Matthews and Wells, Science 260:1113, 1993); positional-scanning peptide libraries (Rano et al., Chem. Biol. 4:149, 1996); and mixture-based peptide libraries (Turk et al., Nature Biotechnology 19:661, 2001). Positional-scanning synthetic peptide libraries are based on the detection of cleavage by the release of a C-terminal fluorogenic group. The technique is rapid and enables analysis of all possible peptide sequences. Currently the libraries only provide sequence specificity N-terminal to the cleavage site and cannot be used with proteases that require amino acid residues C-terminal to the cleavage site; this restricts their use largely to serine and cysteine proteases. Mixture-based peptide libraries offer an alternative that can provide both general applicability and speed. The cleavage site motif C-terminal to the cleavage site is first determined by partial digestion and N-terminal sequencing of a completely random peptide mixture. Information from this first round of screening is then used to design a second library in which strong selected amino acids are fixed, allowing data on sites N-terminal to the cleavage site to be obtained. Reiteration of this process allows an optimal recognition sequence to be determined. The method has recently been used to determine the cleavage motifs of a variety of matrix-metalloproteinases (Turk et al., Nature Biotechnology 19:661, 2001). The phage display method has been used to determine peptide substrates for a number of proteases, for example, plasmin (Hervio et al., Chemistry & Biology 7:443, 2000); tissue-type plasminogen activator (Ding et al., Proc. Natl. Acad. Sci USA 92: 7627, 1995; Ke et al., J. Biol. Chem. 272:16603, 1997); prostate-specific antigen (Coombs et al., Chemistry & Biology 5:475, 1998); and membrane type-1 matrix metalloproteinase (Ohkubo et al., Biochem. Biophys. Res. Commun. 266:308, 1999). The methodology for substrate phage display is illustrated in FIG. 2. Each phagemid codes for a phage coat protein and a tether with a protease substrate sequence linking the two. To create a phage library, the substrate sequence is randomized by site-directed mutagenesis. Each fusion protein is displayed on the surface of a phage particle. For screening, the phage particles are incubated with the protease of interest. The entire digest is then captured using a support with affinity for the tether. Phage with digestion resistant sequences bind the support via the tether. The tether can be an epitope for monoclonal antibody, a histidine tag or a protein-binding peptide. Phage with labile peptide sequences lack the tether and are not bound by the support. Phage with resistant sequence are subsequently released under elution conditions that disrupt binding between the tether and affinity support. Phage with desired sequences, either labile or resistant to the protease of interest, are propagated in bacteria and the cycle is repeated for enrichment. The incubation conditions can be changed to vary the stringency of the screening criteria. For example, highly labile sequences can be identified by selecting for phage that are released at lower protease concentrations or after shorter incubation times.

For the purposes of the present invention, sequences which are labile to target enzyme but resistant to serum proteins can be preferentially selected from a library of peptides. For example, in a first screen, phage that are released upon incubation with the target enzyme are enriched. These phage are then incubated with serum proteins. Phage which remain on the affinity support are then enriched. The DNA sequence of the phagemids can be determined and translated into an amino-acid sequence.

One potential disadvantage of the phage display screening technique is that the peptides may adopt a different conformation when displayed on the phage surface. The peptides may therefore lose their specificity when used as a linker in the polymer-drug conjugate. Hence it may be necessary to study the kinetics of cleavage of the screened peptides in conjugation with a polymeric carrier of choice, in order to confirm their specificity towards the target enzyme.

For example, in order to evaluate whether a particular linker is suitable for use in an inventive conjugate, a set of polymer-linker-drug conjugates or polymer-linker-dye conjugates may be synthesized for kinetic analysis. Without limitation, one could synthesize mPEG-linker-pNA conjugates (where MPEG is methoxy poly(ethyleneglycol) and pNA is the dye p-nitroanilide). Once the conjugates have been prepared, the kinetics of enzymatic cleavage are readily determined. Preferred conjugates do not release a significant amount of active drug molecules in the blood circulation. The stability of the test conjugates in circulation may be assessed by incubating these with serum (e.g., as described in Trouet, Masquelier et al., Proc. Natl. Acad. Sci. USA 79:626-629, 1982). The digestion mixtures are then analyzed by size exclusion HPLC which separates large polymers from small dye or drug molecules. Dye or drug molecules that have been leaked are detected using a spectrophotometer or fluorometer.

The linker sequences are also preferably cleaved at much higher rates by the target enzyme than by non-specific enzymes in normal tissues. Fulfilling this condition is preferred in order to ensure site-specific release of the active drug molecules. To evaluate the specificity of cleavage, the test conjugates are first incubated with the target enzyme(s) and the initial rates of release are measured. A similar experiment is then performed with the non-specific enzymes.

The kinetics can be modeled using the Michaelis-Menten equation, that is,

$$\frac{1}{V i} = \frac{1}{{k}_{c ot} {E}_{total}} + ( \frac{{K}_{m}}{{k}_{ca t} {E}_{total}} ) \frac{1}{{S}_{o}}$$

where Vᵢ is the initial reaction velocity, Sₒ is the substrate concentration, presumed to remain relatively constant during the early reaction course, Eₜₒₜₐₗ is the enzyme concentration, including both bound and non-bound forms, Kₘ is the Michaelis-Menten constant and k_{cat} is the turnover rate of the substrate-enzyme complex to product. A double reciprocal plot (of 1/Vᵢ versus Sₒ) will yield the kinetic constants. The ratio k_{cat}/Kₘ is widely used in the literature to characterize the efficiency of enzymatic cleavage. The difference in the k_{cat}/Kₘ for target enzyme and non-specific enzymes gives a measure of the specificity of the peptide sequence for a particular enzyme. A good candidate for a linker will be cleaved at least 2, 5, 10, or 50 times more efficiently by the target enzyme than by non-specific enzymes (e.g., without limitation serum proteases).

In certain embodiments the linker can also include spacer segments that increase the distance between the bulky polymeric carrier and the drug molecule. For example, a spacer segment can be placed between the cleavage recognition sequence and the polymeric carrier and/or between the cleavage recognition sequence and the drug component. As further explained in the Examples below, an increase in distance between the bulky polymeric carrier and the cleavage recognition sequence may reduce steric hindrance between the digestive enzyme and polymeric carrier and hence facilitate cleavage of the linker. The spacer segment(s) may have any chemical composition. In certain embodiments they can be constructed from a polymer, e.g., without limitation a synthetic polymer such as poly(ethylene glycol) or a natural oligosaccharide or oligopeptide (i.e., whereby additional amino acid or sugar residues are added on either side of the recognition segment). The spacer segments may also result from the specific covalent or non-covalent means used to associate the polymeric carrier, linker and drug components. For example, the use of a ligand/receptor pair to associate the polymeric carrier with the recognition segment instead of a direct covalent bond will necessarily increase the distance between the two components. Preferably a spacer segment is not susceptible to cleavage by the digestive enzyme of interest. A spacer segment may be modified to increase its stability to non-specific enzymatic digestion. For example, for an oligopeptide spacer these modifications may include cyclization of the peptide, incorporation of D-amino acids, etc.

It is also worth noting that unless the drug molecule is associated with the linker at the cleavage site, the cleavage product will necessarily include certain additional residues as compared to the free drug molecule. Under certain circumstances these additional residues may interfere with the biological activity of the drug. Accordingly, it may prove advantageous to select an attachment site on the drug molecule for the linker that minimizes any such reduction in biological activity. These sites are commonly referred to as “bulk tolerant” sites. In addition, it may prove advantageous to optimize the number of residues that lie between the drug molecule and the cleavage site of the linker. Note that any reduction in the number or residues may reduce the specificity of the recognition segment for the digestive enzyme. Accordingly, there may be a need to balance digestion rate, specificity of cleavage and maintenance of biological activity. Methods and assays for performing suitable optimizations are further described in the Examples.

As mentioned earlier, the linkers of the present invention need not necessarily include a digestible oligopeptide segment. For example, the linkers may alternatively include digestible oligosaccharide sequences. Again the chemical composition of the recognition segment (e.g., the length and sequence of sugar residues) will depend for the most part on the motif that is recognized and cleaved by the digestive enzyme of interest. While the cleavage motifs of glycosidases have not been characterized in as much detail as those of proteases, a number of general synthetic routes have been developed in recent years for preparing specific oligosaccharides. Chemical coupling methods have become increasingly sophisticated to fine-tune reactivity of reagents by fortuitous choices of anomeric activating group and protecting groups. As a result, oligosaccharide synthesis has become more predictable and reliable and a number of oligosaccharide libraries have been synthesized in solution and on solid phases (Flitsch, Curr. Opin. Chem. Biol. 4:619, 2000). An exemplary glycosidase in this category is heparanase. Heparanase is an endo-β-D-glucuronidase that catalyzes the hydrolytic cleavage of the β-1,4-glycosidic bond between a D-glucuronate and a D-glucosamine in heparan sulfate (Pikas et al., J. Biol. Chem. 273:18770, 1998; Jemth et al., J. Biol. Chem. 277:30567, 2002). Interestingly, for the purposes of the present invention, heparanase has been implicated in many important physiological and pathological processes including tumor cell metastasis, angiogenesis and leukocyte migration (Vlodavsky et al., Israel Medical Association Journal, 2:37, 2000; Zcharia et al., Journal of Mammary Gland Biology and Neoplasia, 6:311, 2001).

Targeting Agents

In certain embodiments the inventive conjugates may be modified to include targeting agents that will direct an inventive conjugate to a particular cell type, collection of cells, or tissue. Preferably, the targeting agents are associated with the polymeric carrier. A variety of suitable targeting agents are known in the art (Cotten et al., Methods Enzym. 217:618, 1993; Torchilin, Eur. J. Pharm. Sci. 11:S81, 2000; Garnett, Adv. Drug Deliv. Rev. 53:171, 2001). For example, any of a number of different materials which bind to antigens on the surfaces of target cells may be employed. Antibodies to target cell surface antigens will generally exhibit the necessary specificity for the target. In addition to antibodies, suitable immunoreactive fragments may also be employed, such as the Fab, Fab′, or F(ab′)₂ fragments. Many antibody fragments suitable for use in forming the targeting mechanism are already available in the art. Similarly, ligands for any receptors on the surface of the target cells may suitably be employed as targeting agent. These include any small molecule or biomolecule, natural or synthetic, which binds specifically to a cell surface receptor, protein or glycoprotein found at the surface of the desired target cell.

Pharmaceutical Compositions

Once the inventive conjugates have been prepared, they may be combined with pharmaceutically acceptable carriers to form a pharmaceutical composition. As would be appreciated by one of skill in this art, the carriers may be chosen based on the route of administration as described below, the location of the target issue, the drug being delivered, the time course of delivery of the drug, etc.

As used herein, the term “pharmaceutically acceptable carrier” means a non-toxic, inert solid, semi-solid or liquid filler, diluent, encapsulating material or formulation auxiliary of any type. Remington 's Pharmaceutical Sciences Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Administration

The pharmaceutical compositions of this invention can be administered to a patient by any means known in the art including oral and parenteral routes. The term “patient”, as used herein, refers to humans as well as non-humans, including, for example, mammals, birds, reptiles, amphibians and fish. Preferably, the non-humans are mammals (e.g., a rodent, a mouse, a rat, a rabbit, a monkey, a dog, a cat, a primate, or a pig). In certain embodiments parenteral routes are preferred since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive conjugate with suitable non-irritating excipients or carriers such as cocoa butter, polyethylene glycol, or a suppository wax which are solid at ambient temperature but liquid at body temperature and therefore melt in the rectum or vaginal cavity and release the inventive conjugate.

Dosage forms for topical or transdermal administration of an inventive pharmaceutical composition include ointments, pastes, creams, lotions, gels, powders, solutions, sprays, inhalants, or patches. The inventive conjugate is admixed under sterile conditions with a pharmaceutically acceptable carrier and any needed preservatives or buffers as may be required. Ophthalmic formulations, ear drops and eye drops are also contemplated as being within the scope of this invention. The ointments, pastes, creams and gels may contain, in addition to the inventive conjugates of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Transdermal patches have the added advantage of providing controlled delivery of a compound to the body. Such dosage forms can be made by dissolving or dispensing the inventive conjugates in a proper medium. Absorption enhancers can also be used to increase the flux of the compound across the skin. The rate can be controlled by either providing a rate controlling membrane or by dispersing the inventive conjugates in a polymer matrix or gel.

Powders and sprays can contain, in addition to the inventive conjugates of this invention, excipients such as lactose, talc, silicic acid, aluminum hydroxide, calcium silicates and polyamide powder, or mixtures of these drugs. Sprays can additionally contain customary propellants such as chlorofluorohydrocarbons.

When administered orally, the inventive conjugates are preferably, but not necessarily, encapsulated. A variety of suitable encapsulation systems are known in the art (“Microcapsules and Nanoparticles in Medicine and Pharmacy,” Edited by Doubrow, M., CRC Press, Boca Raton, 1992; Mathiowitz and Langer J. Control. Release 5:13, 1987; Mathiowitz et al., Reactive Polymers 6:275, 1987; Mathiowitz et al., J. Appl. Polymer Sci. 35:755, 1988; Langer Acc. Chem. Res. 33:94,2000; Langer J. Control. Release 62:7, 1999; Uhrich et al., Chem. Rev. 99:3181, 1999; Zhou et al., J. Control. Release 75:27, 2001; and Hanes et al., Pharm. Biotechnol. 6:389, 1995). Preferably the inventive conjugates are encapsulated within biodegradable polymeric microspheres or liposomes. Examples of natural and synthetic polymers useful in the preparation of biodegradable microspheres include carbohydrates such as alginate, cellulose, polyhydroxyalkanoates, polyamides, polyphosphazenes, polypropylfumarates, polyethers, polyacetals, polycyanoacrylates, biodegradable polyurethanes, polycarbonates, polyanhydrides, polyhydroxyacids, poly(ortho esters) and other biodegradable polyesters. Examples of lipids useful in liposome production include phosphatidyl compounds, such as phosphatidylglycerol, phosphatidylcholine, phosphatidylserine, phosphatidylethanolamine, sphingolipids, cerebrosides and gangliosides.

Pharmaceutical compositions for oral administration can be liquid or solid. Liquid dosage forms suitable for oral administration of inventive compositions include pharmaceutically acceptable emulsions, microemulsions, solutions, suspensions, syrups and elixirs. In addition to an encapsulated or unencapsulated conjugate, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions can also include adjuvants, wetting agents, emulsifying and suspending agents, sweetening, flavoring and perfuming agents. As used herein, the term “adjuvant” refers to any compound which is a nonspecific modulator of the immune response. In certain preferred embodiments, the adjuvant stimulates the immune response. Any adjuvant may be used in accordance with the present invention. A large number of adjuvant compounds is known in the art (Allison, Dev. Biol. Stand. 92:3, 1998; Unkeless et al., Annu. Rev. Immunol. 6:251, 1998; and Phillips et al., Vaccine 10: 151, 1992).

Solid dosage forms for oral administration include capsules, tablets, pills, powders and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol and silicic acid, (b) binders such as, for example, carboxymethylcellulose, alginates, gelatin, polyvinylpyrrolidinone, sucrose and acacia, (c) humectants such as glycerol, (d) disintegrating agents such as agar-agar, calcium carbonate, potato or tapioca starch, alginic acid, certain silicates and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art.

It will be appreciated that the exact dosage of the inventive conjugate is chosen by the individual physician in view of the patient to be treated. In general, dosage and administration are adjusted to provide an effective amount of the inventive conjugate to the patient being treated. As used herein, the “effective amount” of an inventive conjugate refers to the amount necessary to elicit the desired biological response. As will be appreciated by those of ordinary skill in this art, the effective amount of inventive conjugate may vary depending on such factors as the desired biological endpoint, the drug to be delivered, the target tissue, the route of administration, etc. For example, the effective amount of inventive conjugate containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy. Long acting pharmaceutical compositions might be administered every 3 to 4 days, every week, or once every two weeks depending on half-life and clearance rate of the particular composition.

The conjugates of the invention are preferably formulated in dosage unit form for ease of administration and uniformity of dosage. The expression “dosage unit form” as used herein refers to a physically discrete unit of conjugate appropriate for the patient to be treated. It will be understood, however, that the total daily usage of the compositions of the present invention will be decided by the attending physician within the scope of sound medical judgment. For any conjugate, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies is used in formulating a range of dosage for human use.

These and other aspects of the present invention will be further appreciated upon consideration of the following Examples, which are intended to illustrate certain particular embodiments of the invention but are not intended to limit its scope, as defined by the claims.

### EXAMPLES

Introduction

The following Examples describe the synthesis and characterization of inventive conjugates that release anti-cancer drugs in the presence of matrix-metalloproteinase II (MMP-2), a widely studied proteinase that is overexpressed in a number of cancers (Woessner and Nagase, Matrix Metalloproteinases and TIMPS, Oxford University Press, 2000). The conjugates include linkers with different fractions of an oligopeptide motif that is recognized and cleaved by MMP-2. The conjugates are characterized by comparing the rate of drug release in the presence of MMP-2 and fetal bovine serum (an in vitro control for a normal circulation environment). The cytotoxicity of the conjugates is also compared by exposing various human tumor cell lines to the cleavage products. In vivo experiments with a mice model are also described.

Expression of MMP-2 has been found to be elevated in a number of human epithelial cancers, including breast (Davies et al., British Journal of Cancer 67:1126, 1993); prostate (Harndy et al., British Journal of Cancer 69:177, 1994); colon (Levy et al., Cancer Research 51:439, 1991); ovary (Naylor et al., International Journal of Cancer 58:50, 1994); bladder (Davies et al., British Journal of Cancer 67:1126, 1993); and gastric carcinoma (d'Errico et al., Mod Pathol. 4:239, 1991). Elevated expression has been found in both malignant epithelial cells and the surrounding stromal fibroblasts. Moreover, MMP-2 has been implicated with the angiogenic and metastatic potential of cancer.

### Example 1

### Synthesis of CM-dextran-oligopeptide-doxorubicin Conjugates

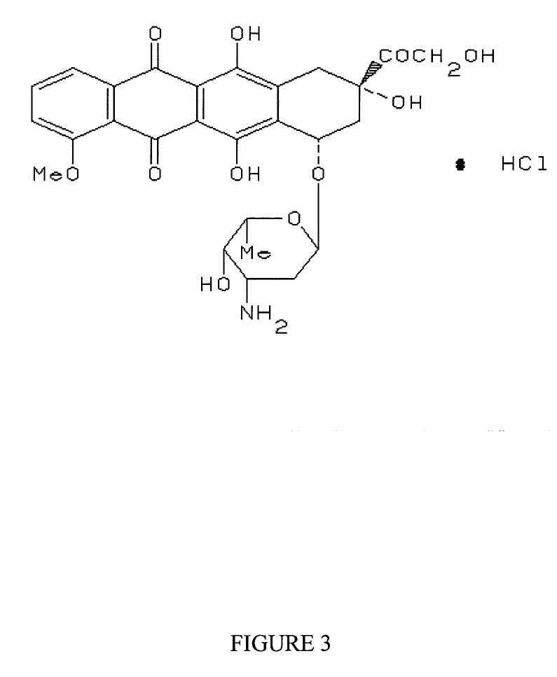

Doxorubicin belongs to the class of anthracyclines which kill cells by intercalating within DNA molecules. The structure of doxorubicin is illustrated in FIG. 3. Free doxorubicin has a clinical dose of 60-80 mg/m². After intravenous injection, the drug molecules distribute ubiquitously throughout the body before being quickly eliminated by renal excretion (Cassidy et al., Cancer Surveys 17:315, 1993). Major toxicity is observed within the haemolymphopoietic system, gastro-intestinal tract, skin, testes and heart. Doxorubicin is used widely in the treatment of cancers, including breast, ovarian, bladder, lung cancers, non-Hodgkin's lymphoma, Hodgkin's disease and sarcoma.

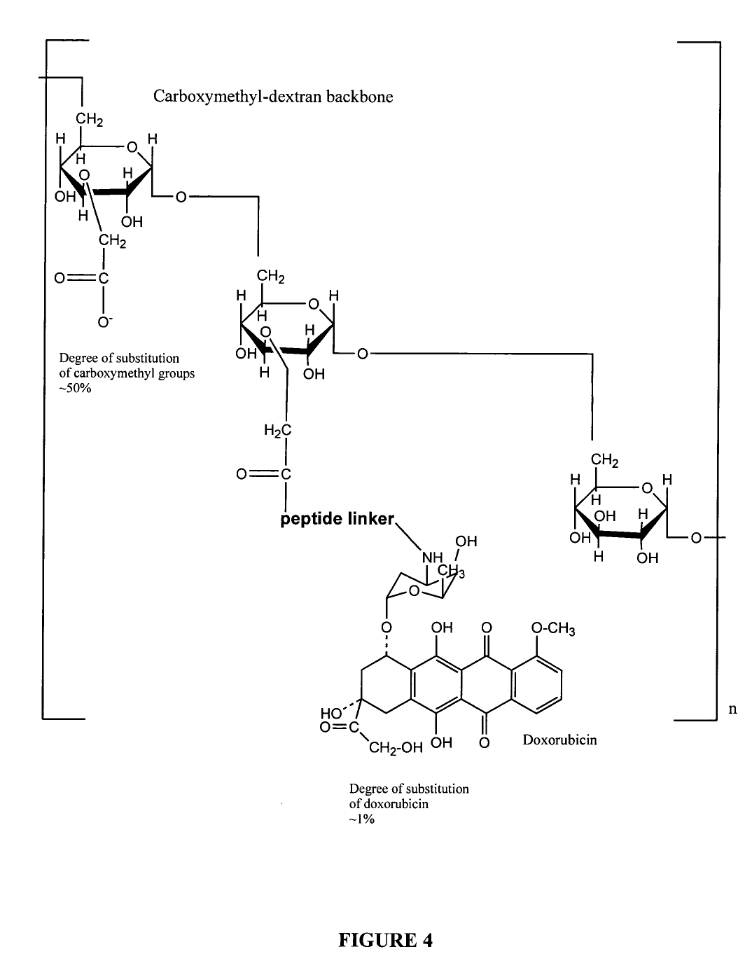

Carboxymethyl-dextran-oligopeptide-doxorubicin conjugates (see FIG. 4), were synthesized using traditional techniques of peptide coupling and dextran modification. Oligopeptides of four to seven amino acids were used as linkers between carboxymethyl-dextran (CM-dextran) and doxorubicin. They are listed below in Table 1. The potential cleavage site is between P1 and P1′ (using the nomenclature of Schechter and Berger, Biochem. Biophys. Res. Comm. 27:157, 1967), wherein residues on the N-terminal side of the cleavage site are unprimed and residues on the C-terminal side of the cleavage site are primed). The four linkers differ solely in the number of amino acids that are on the C-terminal side of the cleavage site.

TABLE 1

| | | | | | | |
|---|---|---|---|---|---|---|
| P4 | P3 | P2 | P1 | P1′ | P2′ | P3′ |

| | | | | | | |
|---|---|---|---|---|---|---|
| Ile | Pro | Val | Gly | Leu | Ile | Gly |
| Ile | Pro | Val | Gly | Leu | Ile | |
| Ile | Pro | Val | Gly | Leu | | |
| Ile | Pro | Val | Gly | | | |
| *Ile | Pro | Val | Ser | Leu | Arg | Ser |

*Sequence obtained from Turk et al., Nature Biotechnology 19:661, 2001.

Carboxymethyl-dextran was prepared by exposing dextran with an average MW of 40,000 Da to chloracetic acid. Dextran was selected in part because of its biocompatibility (Sgouras and Duncan, J. Mater. Sci.: Mat. Med. 1:61, 1990) and biodegradability (Vercauteren, Schacht et al., J. Bioactive and Compatible Polymers 7:346 1992). In addition, from a synthetic standpoint, the hydroxyl groups on the dextran backbone provide convenient sites for covalent association with the oligopeptide recognition segment.

The oligopeptides were synthesized by conventional solid-phase techniques (Bodansky and Bodansky, The Practice of Peptide Synthesis, Springer 1994) and N-terminal protected with an FMOC group. The N-terminal protected FMOC-peptides (2 eq.) were conjugated with doxorubicin (1 eq.) in the presence of N,N″-diisopropylethylamine (5 eq.) in DMF using PyBop (1.9 eq.) and HOBT (2 eq.) as coupling reagents. The reactions were held at room temperature overnight. From a synthetic standpoint, the aminoribosyl group on doxorubicin allows the drug molecule to be covalently associated with the oligopeptide linker via an aminolysis reaction. Reversed-phase chromatography employing an acetonitrile gradient in 0.2% trifluoroacetic on a Vydac C18 column was used to isolate the FMOC-oligopeptide-doxorubicin. Purity as analyzed by HPLC was greater than 95%. Deprotection of FMOC was completed in 5 minutes using 10% piperidine in DMF followed by quenching on ice with a mixture of trifluoroacetic acid/pyridine/DMF (3:7:20). After deprotection, oligopeptide-doxorubicin was covalently linked to pre-activated dextran carrier in the presence of N-ethoxycarbonyl-2-ethoxy-1,2-dihydroquinoline (EEDQ).

Size exclusion chromatography on a Sephadex G25 column was then used to separate carboxymethyl-dextran-oligopeptide-doxorubicin conjugates from unreacted oligopeptide-doxorubicin complexes. Conjugate purity as analyzed by HPLC was greater than 99%. The loading density of the conjugate was about 1%.

The oligopeptide sequences in Table 1 were based on the MMP-2 cleavage motifs that have been determined using mixture-based oriented peptide libraries (Turk et al., Nature Biotechnology 19:661, 2001). The optimal MMP-2 cleavage motif determined by Turk et al., was Ile-Pro-Val-Ser-Leu-Arg-Ser (P4 through P3′, shown on the bottom of Table 1). However, the presence of reactive side chains on serine and arginine (i.e., at positions P1, P2′ and P3′, shown in bold in Table 1) and the fact that doxorubicin is labile to harsh deprotection procedures, posed problems for synthesis. As a result, the second best amino acids at positions P1, P2′ and P3′ with non-reactive side chains were used in synthesizing the inventive carboxymethyl-dextran-oligopeptide-doxorubicin conjugates (i.e., an Ile-Pro-Val-Gly-Leu-Ile-Gly motif was selected instead).

### Example 2

### Peptidyl Doxorubicin Release in the Presence of MMP-2

Two opposing factors determine the optimization of the length of an oligopeptide recognition segment, namely the cytotoxicity of the released drug molecule and the sensitivity of the linker to cleavage by the digestive enzyme. If doxorubicin is linked to the oligopeptide adjacent to the cleavage site (i.e., at the P1′ position), the released molecule is free doxorubicin—a fully active drug. The cytotoxicity may probably be attenuated if doxorubicin is linked further away from the cleavage site (e.g., at the P2′ or P3′ positions). However, past studies on matrix-metalloproteinase specificity have implied that substrate sites from P3 to P3′ are all required for cleavage to occur.

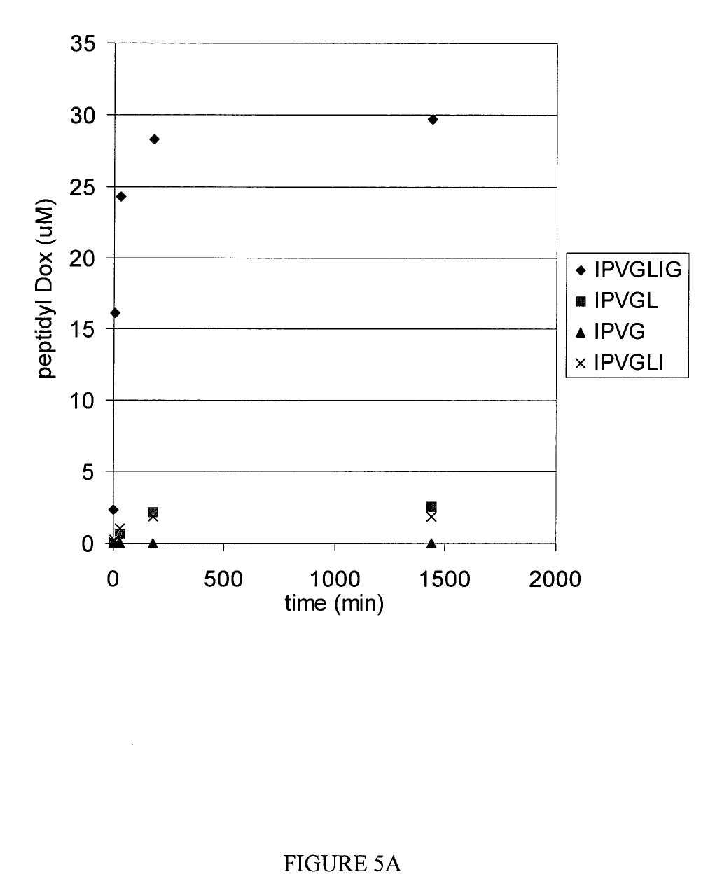

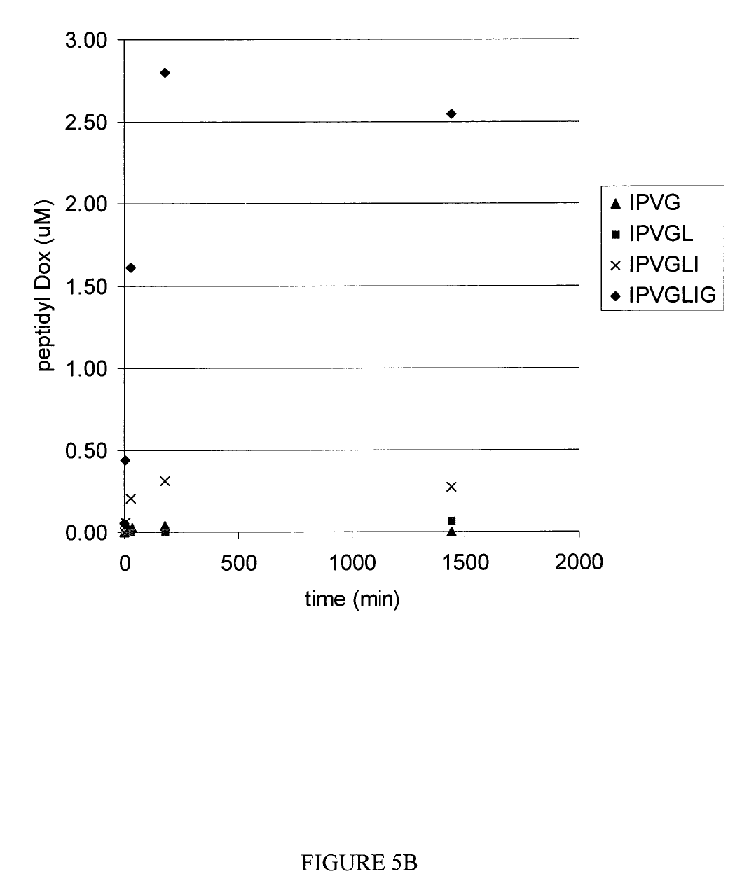

Enzymatic release of doxorubicin from the conjugates prepared in Example 1 was therefore measured. The conjugates were incubated with purified active MMP-2 and the results are illustrated in FIG. 5A (high enzyme and substrate concentrations) and FIG. 5B (low enzyme and substrate concentrations). Under both sets of conditions, the rate of doxorubicin release followed the trend IPVGLIG>>IPVGLI>IPVGL>IPVG, i.e., in agreement with the literature suggesting that substrate positions from P3 to P3′ are important for MMP recognition and cleavage. Conjugates with the shortest oligopeptide (IPVG) were insensitive to the presence of MMP-2 even under conditions with high enzyme and high substrate concentrations. In a control set-up wherein PBS was added instead of MMP-2, no detectable release was observed for all conjugates confirming that release was triggered by MMP-2.

In order to further characterize the enzymatic cleavage of the linker IPVGLIG, the rate constant for cleavage was also determined according to the Michaelis-Menten model, i.e., based on the equilibrium model:

$$E + S {{⟷}_{{k}_{- 1}}}^{{k}_{1}} E S {⟶}^{{k}_{c a t}} E + P$$

where Kₘ=k₋₁/k₁ is termed the Michaelis-Menten constant, k_{cat} is the turnover rate constant and k_{cat}/Kₘ is termed the specificity constant and provides a measure of the rate of enzymatic cleavage.

As shown in Table 2, despite the attachment of doxorubicin the rate of enzymatic cleavage for IPVGLIG-doxorubicin compares favorably with that of the optimal cleavage motif IPVSLRSG (Turk et al., Nature Biotechnology 19:661, 2001). However, when IPVGLIG-doxorubicin was covalently attached to carboxymethyl-dextran, the specificity constant decreased by two orders of magnitude. This suggested that a strong steric hindrance effect was being imposed by the polymer backbone.

TABLE 2

| Species | Structure | k_{cat}/Kₘ (M⁻¹ · s⁻¹) |
|---|---|---|
| Optimal Linker* | IPVSLRSG | 0.82 ± 0.06 × 10⁵ |
| Linker-Drug | IPVGLIG-doxorubicin | 1.40 × 10⁵ |
| Polymer-Linker-Drug | CM-dextran-IPVGLIG- | 1.77 × 10³ |
| | doxorubicin | |

*Value obtained from Turk et al., Nature Biotechnology 19:661, 2001.

As shown in Table 3, when ionic strength was increased under digestion conditions, the specificity constant increased. This trend suggested that at least part of the hindrance was due to electrostatic repulsion between the negatively-charged polymer backbone and the active site of MMP-2. These results indicate that the sensitivity of the polymer-linker-drug conjugate towards MMP-2 should be improved by decreasing the charge density of the polymer-backbone. This is further demonstrated in Example 8.

TABLE 3

| Salt concentration (NaCl, M) | k_{cat}/Kₘ (M⁻¹ · s⁻¹) |
|---|---|
| 0.01 | 7.50 × 10¹ |
| 0.10 | 1.15 × 10² |
| 0.20 | 1.09 × 10³ |
| 0.50 | 4.47 × 10³ |

### Example 3

### Stability of CM-dextran-oligopeptide-doxorubicin Conjugates in Serum

To achieve efficient targeting, it is preferable that premature release of active drug molecules in the blood stream is minimal. To this end, the CM-dextran-oligopeptide-doxorubicin conjugates of Example 1 were incubated at 37 C with fetal bovine serum for up to 24 hours. All four conjugates were relatively stable in the serum, exhibiting less than 5% release of doxorubicin.

### Example 4

### In Vitro Cytotoxicity of Peptidyl Doxorubicin

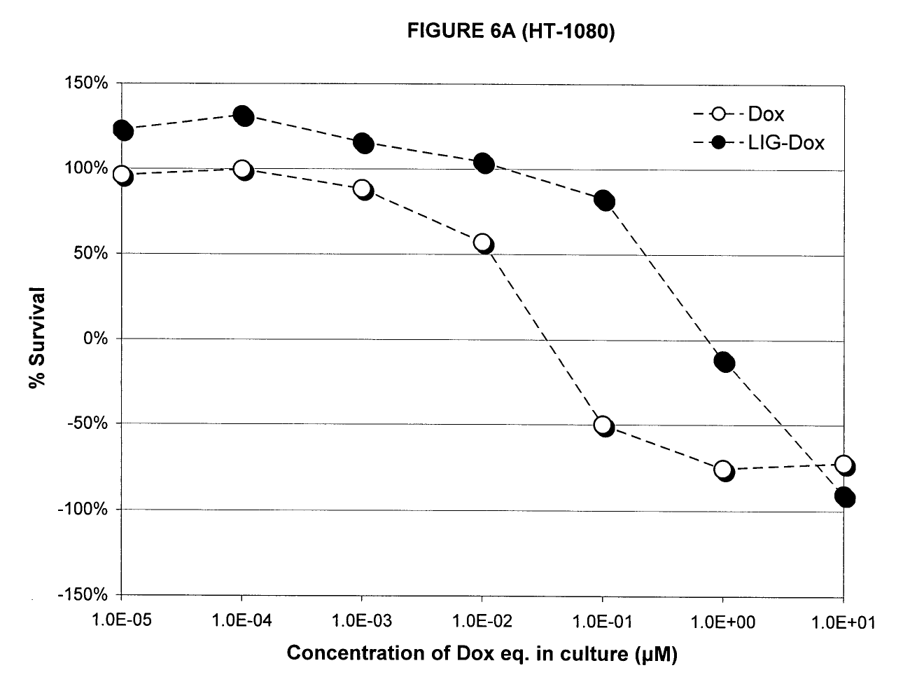

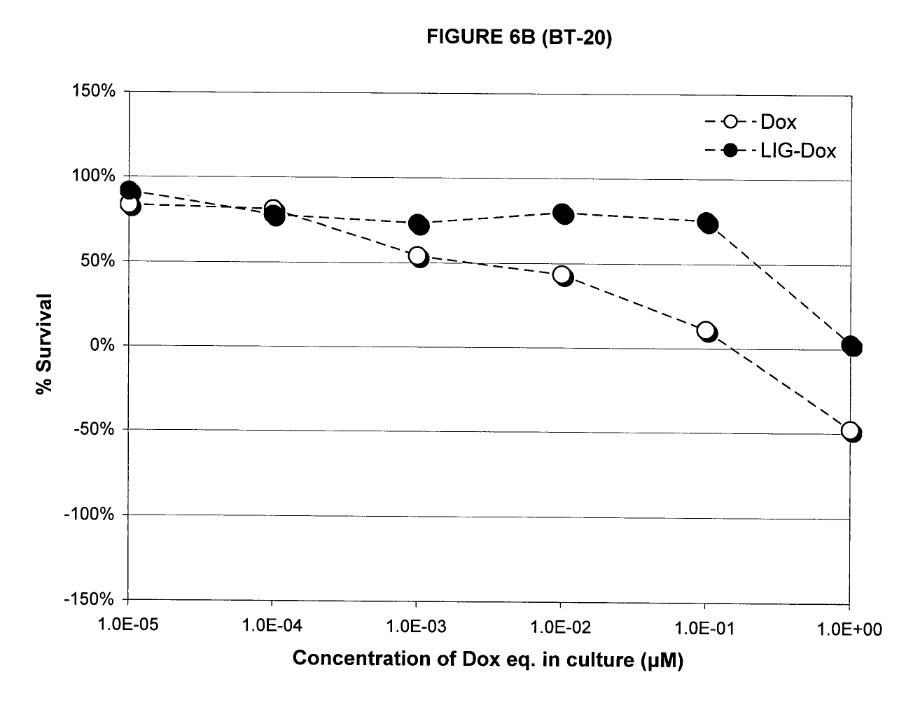

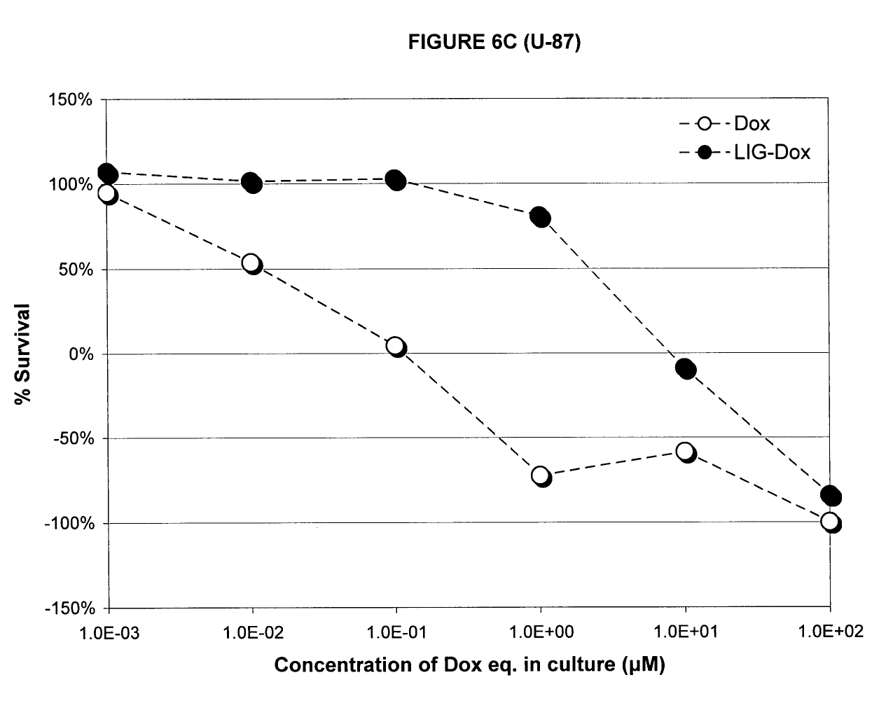

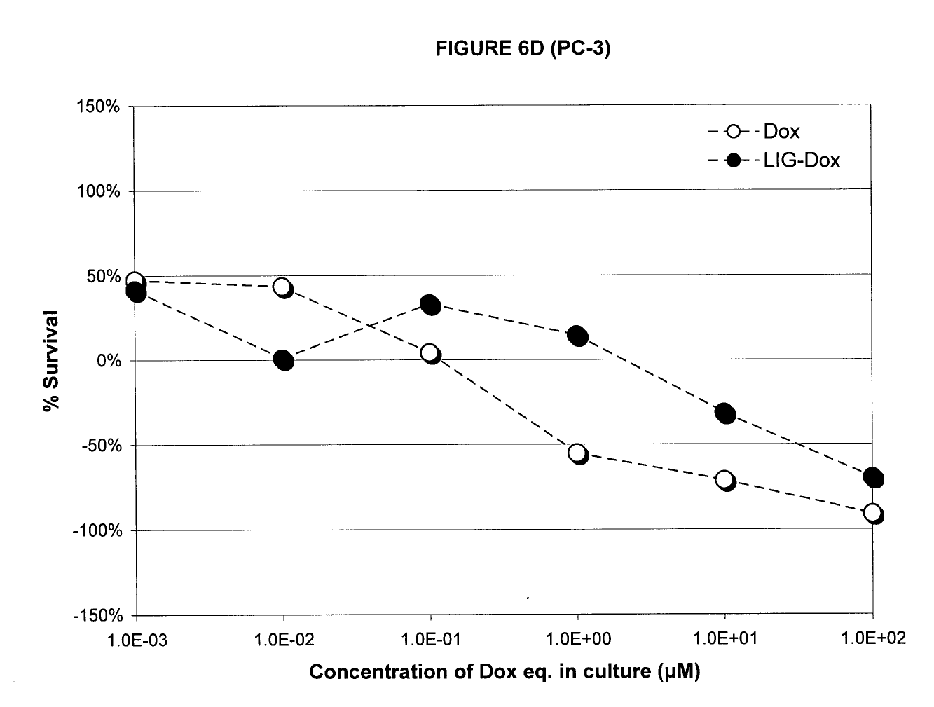

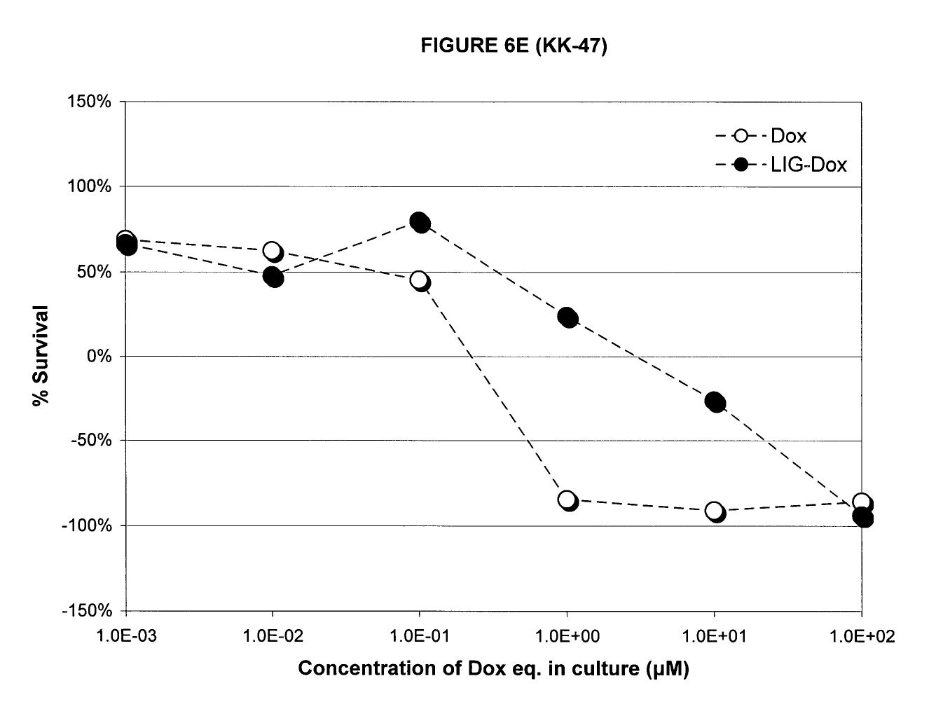

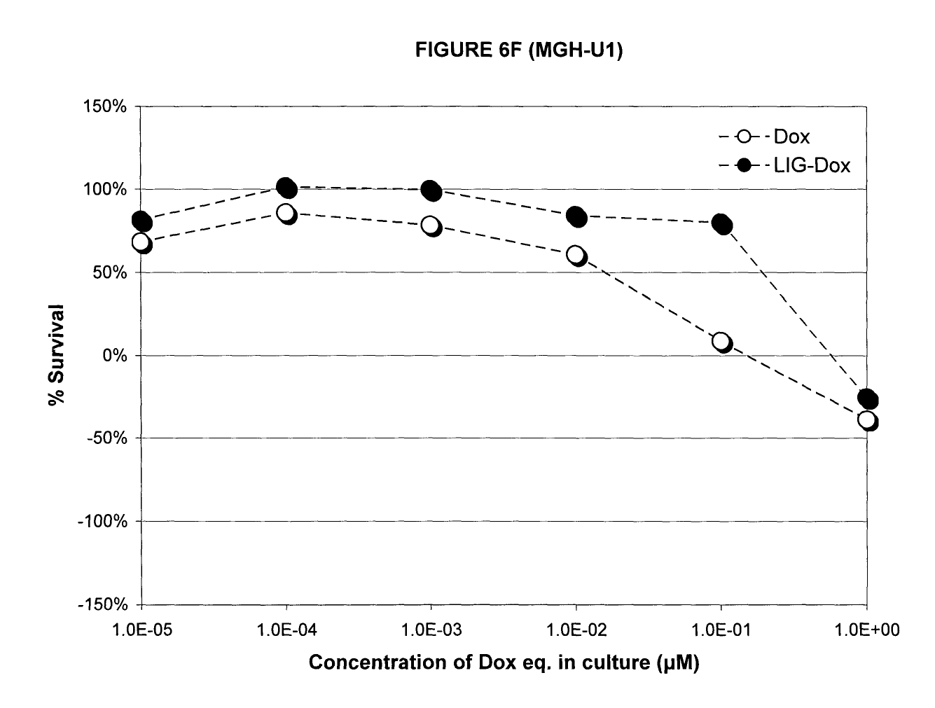

The cytotoxicity of the cleavage product from CM-dextran-IPVGLIG-doxorubicin (i.e., the conjugate most sensitive to cleavage by MMP-2 in this series) was measured. Confirmed by mass spectroscopy to be LIG-doxorubicin, the cleavage product was cytotoxic to a number of human tumor cell lines, namely HT-1080 (a human fibrosarcoma line, FIG. 6A), BT-20 (a human breast carcinoma line, FIG. 6B), U-87 (a human glioblastoma cell line, FIG. 6C), PC-3 (a human prostate tumor cell line, FIG. 6D), KK-47 (a human bladder tumor cell line, FIG. 6E) and MGH-U1 (a human bladder cell line, FIG. 6F). Open circles in FIGS. 6A-F represent free doxorubicin and closed circles represent LIG-doxorubicin.

Cell survival was assayed using an indirect colorimetric immunoassay (MTT assay, e.g., see Alley et al., Cancer Res. 48:589, 1988). The tetrazolium salt MTT is metabolized by NAD-dependent dehydrogenase to form a colored reaction product and the amount of dye formed directly correlates with the number of viable cells. The formula for calculating % survival is given by:

%survival=(Tᵢ−T_{z})/(C−T_{z}) if Tᵢ≧T_{z} or

%survival=(Tᵢ−T_{z})/T_{z} if Tᵢ<T_{z}

where Tᵢ=Net MTT reading of test growth in the presence of drugs; T_{z}=Net MTT reading at time zero with respect to the drug test; and C=Net MTT reading of control growth (see Grever et al., Seminars in Oncology, 19:622, 1992 and Boyd and Paull, Drug Development Research 34:91, 1995).

Overall, LIG-doxorubicin was less potent than the free drug doxorubicin. Doxorubicin exerts its cytotoxicity by intercalating DNA and the ribosyl amino group is important in stabilizing DNA binding through hydrogen bonding. Modification of this amino group may therefore explain the reduction in activity.

### Example 5

### Synthesis of a Dextran-poly(ethyleneglycol)-oligopeptide-doxorubicin Conjugate

The following describes the synthesis of a slightly different conjugate that also includes the drug doxorubicin. The polymeric backbone is dextran grafted with poly(ethyleneglycol) (PEG). Doxorubicin is linked to PEG via a cleavable oligopeptide sequence. Like dextran, PEG is biocompatible (Sgouras and Duncan, J. Mater. Sci.: Mat. Med. 1:61, 1990). However, unlike dextran it is not biodegradable and hence the use of this polymer is limited to a size below the renal excretion limit (Yamaoka et al., J. Pharm. Sci. 83:601, 1994). Pegylation is known to prolong circulation time in the blood stream, reduce immunogenicity and increase solubility. These advantages are highly dependent on the hydrophilicity imparted by the polymer. With these benefits, PEG-L-asparaginase has been approved for clinical use in the United States to treat leukemia (Keating et al., Leukemia Lymphoma 10:153, 1993).

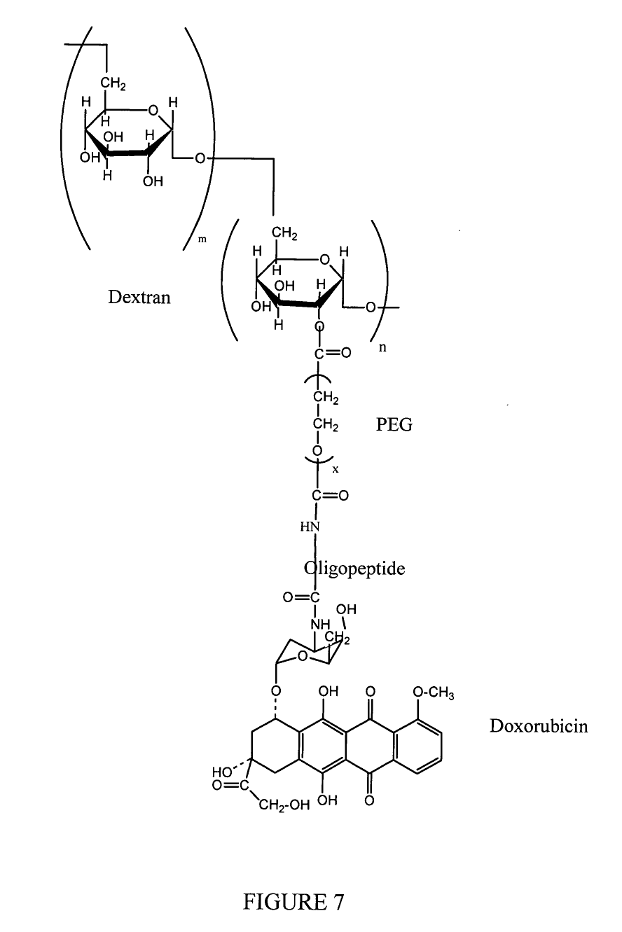

The oligopeptide linkers are synthesized by conventional solid-phase techniques (Bodansky and Bodansky, The Practice of Peptide Synthesis, Springer 1994) and the N-terminus is protected by FMOC. The ribosyl amino group of doxorubicin is condensed with the carboxyl terminus of the oligopeptide in the presence of PyBop and HOBT in DMF. The FMOC protection group on the oligopeptide is then removed using 10% piperidine to expose the free N-terminus, which is then attached to a heterogeneously substituted bivalent PEG-FMOC-NH-PEG-NHS- using EDC as the conjugating reagent in aqueous medium. After conjugation, the FMOC group on PEG is removed using 10% piperidine to afford the resulting intermediate NH₂-PEG-oligopeptide-doxorubicin. In the presence of EDC in aqueous phase, this intermediate is covalently linked via amide linkage to carboxymethyl dextran (prepared as in Example 1) forming a CM-dextran-(PEG-oligopeptide-doxorubicin)ₙ complex. Subsequently, the remaining carboxymethyl groups on dextran are reacted with excessive amount of mPEG-NH₂ using EDC as conjugating reagent. The chemical structure of the conjugate is illustrated in FIG. 7.

As noted above, the ratio of PEG to PEG-oligopeptide-doxorubicin on the dextran core may affect the relative rates at which the final conjugate is engulfed by cells or cleaved by digestive enzymes in the extracellular space. Indeed, the surface hydrophobicity of the conjugate may have a bearing on the endocytosis rate of the conjugate. Hydrophobic particles are generally engulfed more quickly than hydrophilic particles. For this reason, micro- and nanoparticles in drug delivery applications are frequently coated with hydrophilic PEG to elongate their circulation by evading the engulfment by the retinoendothelial system (RES).

With dextran and PEG as the polymer backbone, the conjugates will have a hydrophilic nature. However, loading of hydrophobic drugs could alter the surface characteristics of the conjugates and increase the clearance by the RES (Kataoka, p. 49 in “Targetable polymeric drugs. Controlled drug delivery: challenges and strategies” Ed. by Park, American Chemistry Society, 1997). The hydrophobicity of conjugates can be compared by measuring the contact angle with water on a surface coated with the conjugates. The endocytotic rate of conjugates can also be measured by placing radioactively labeled conjugates in a Kupffer cell culture. Kupffer cells are chosen since they are the major phagocytes in the RES. At specified time points, cell samples are removed, washed extensively and homogenized. The amount of conjugate engulfed is then proportional to the radioactivity detected by a scintillation counter.

### Example 6

### Synthesis and Characterization of a methoxy-poly(ethyleneglycol)-oligopeptide-doxorubicin Conjugate

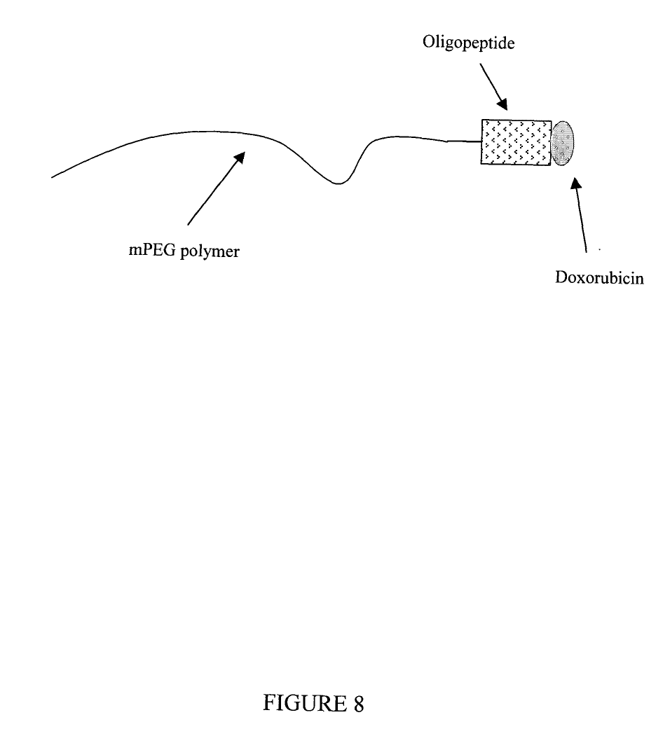

An IPVGLIG-doxorubicin complex was prepared following the procedures described in Example 1 and then covalently attached to a linear flexible polymer, namely methoxy-poly(ethyleneglycol)-NHS ester (mPEG-NHS, nominal MW of ˜20,000 Da from Shearwaters Polymer) in the presence of N,N″-diisopropylethylamine in acetonitrile. The reaction was held overnight at room temperature. The conjugate methoxy-poly(ethyleneglycol)-oligopeptide-doxorubicin was purified by reversed phase chromatography over an acetonitrile gradient in 0.2% trifluoroacetic acid using a Vydac C 18 column. A schematic representation of this conjugate is shown in FIG. 8. The digestion kinetics of mPEG-IPVGLIG-doxorubicin conjugate by MMP-2 was measured and found to be equivalent to that of IPVGLIG-doxorubicin complex, thus much faster than that of CM-dextran-IPVGLIG-doxorubicin conjugate (see Example 2).

The three-dimensional structure of the catalytic domain of MMP-2 was examined (available online in the Brookhaven protein bank). The substrate binding site consists of a groove that is enclosed in the enzyme interior and has space for about six amino acids. Although the two ends of the groove are exposed, directly tethering the recognition segment to a large polymer backbone is likely to give rise to significant steric hindrance. Comparing the results of CM-dextran-IPVGLIG-doxorubicin and mPEG-IPVGLIG-doxorubicin suggests that incorporation of a flexible spacer (e.g., polyethylene oxide) between the digestible oligopeptide and the polymer backbone (e.g., dextran or CM-dextran) may improve release of drugs from such conjugates. Alternatively, these results suggest that one could construct a conjugate using a PEG star polymer with oligopeptide-drugs linked to the outer ends for better presentation of the digestible oligopeptide.

### Example 7

### Methotrexate as an Alternative to Doxorubicin

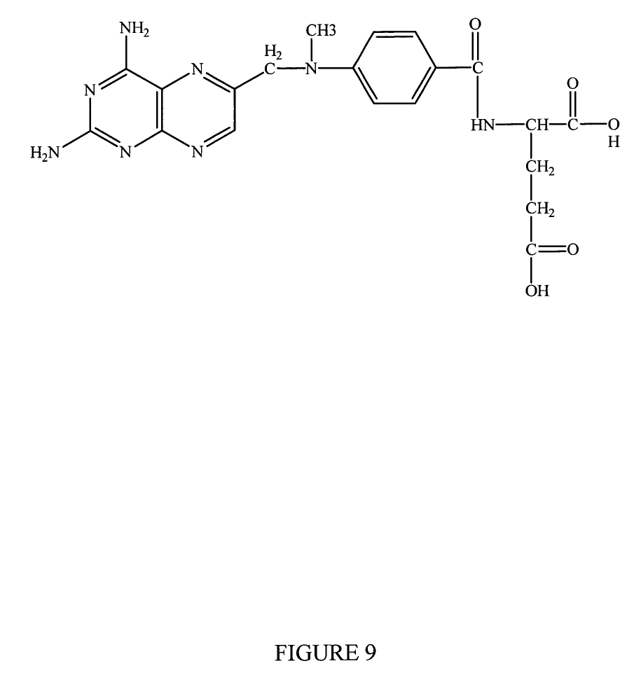

As described earlier, in certain preferred embodiments the activity of a drug that is to be incorporated into a conjugate of the present invention is minimally affected by the covalent attachment of several amino acids (i.e., the peptidyl remnants of linker cleavage). In this context, Rosowsky has reported that methotrexate (an anti-metabolite that is used to treat cancer and arthritis) bears “bulk tolerance” at the y-carboxyl of its glutamic acid (Rosowsky et al., J. Med. Chem. 24:1450, 1981). Methotrexate is effective against a number of human tumors, for example, lymphoblastic leukemia in children, chriocarcinoma and related trophoblastic tumors in women, osteosacroma and carcinomas of breast, head, neck, ovary and bladder. The major side effect is toxicity in gastrointestinal tract and bone marrow (Chamber et al., p. 1389 in “Goodman and Gilman's: The Pharmacological Basis of Therapeutics”, Ed. by Hardman et al., McGraw-Hill, 2001). The structure of methotrexate is illustrated in FIG. 9.

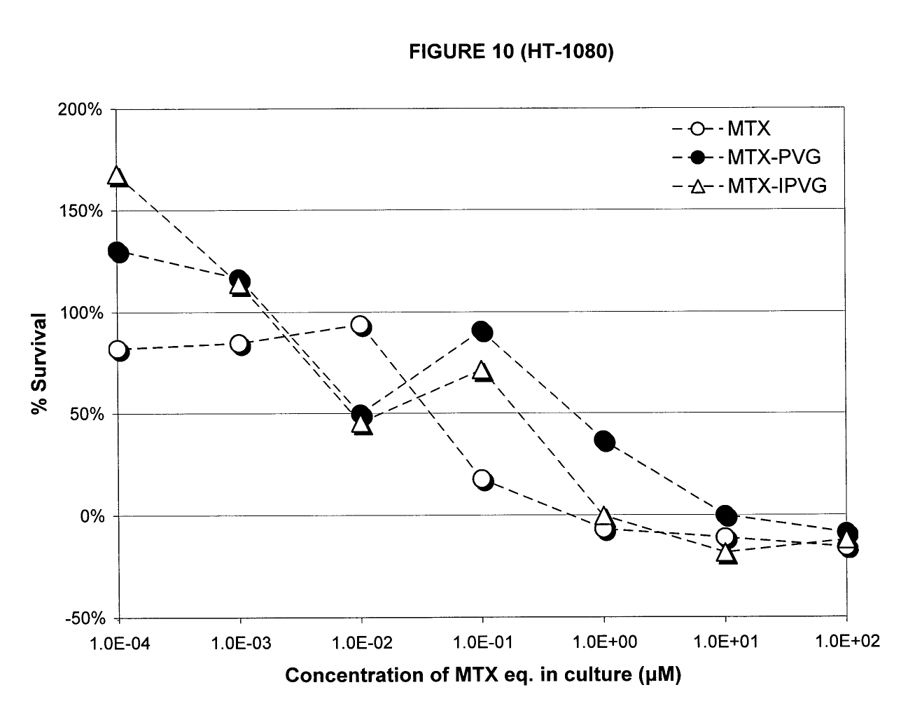

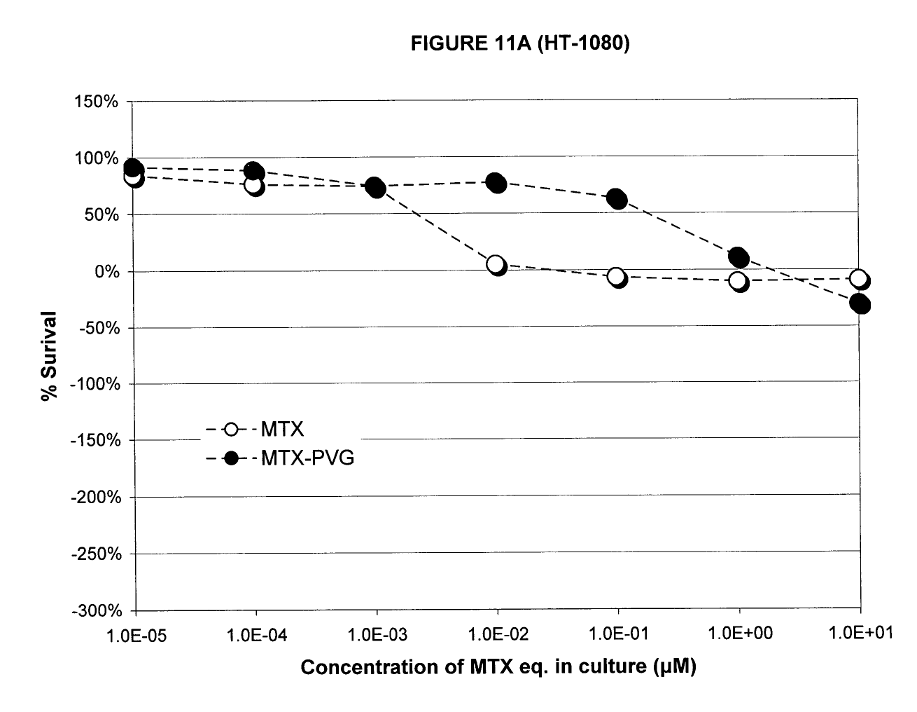

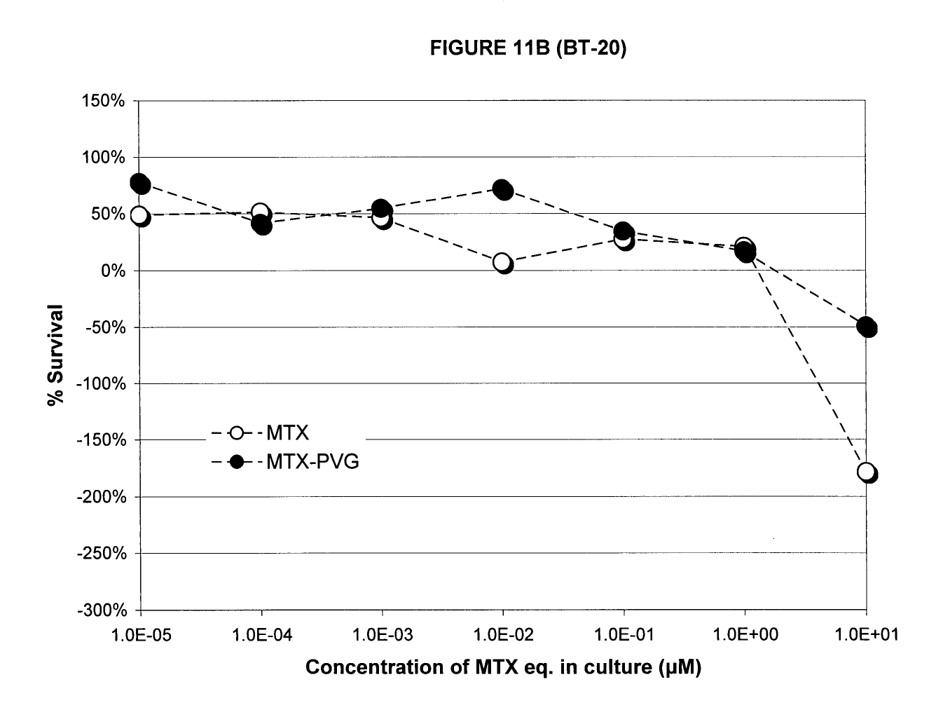

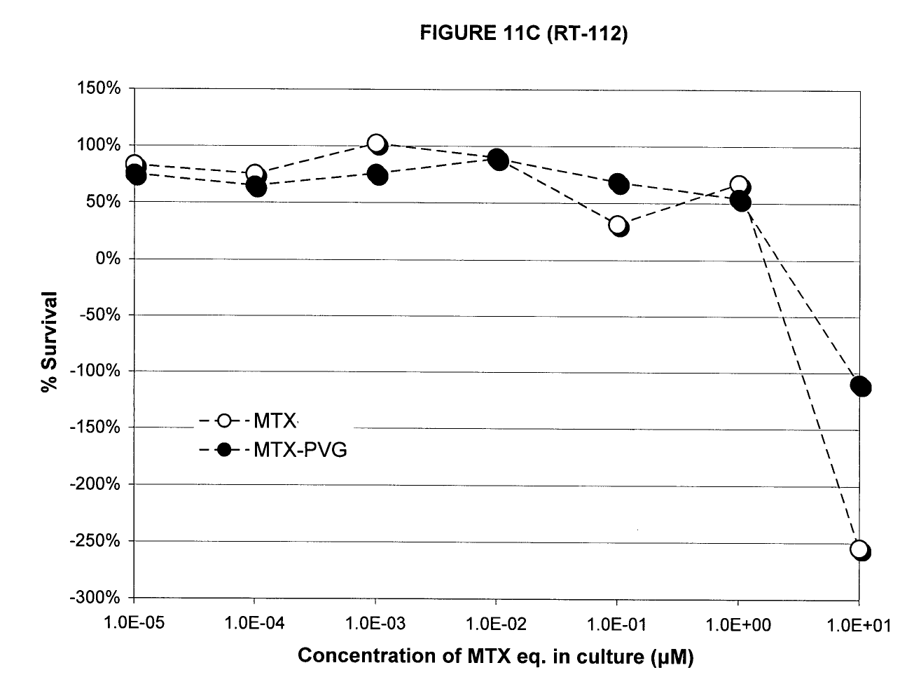

The cytotoxicity of methotrexate and various peptidyl-methotrexates were compared in order to assess bulk tolerance. The peptidyl-methotrexates were prepared by linking short peptides of three to four amino acids to methotrexate. The peptides were chosen to represent appropriate cleavage fragments from the MMP-2 sensitive substrate peptide IPVGLIG that was characterized in Examples 1-2. Methotrexate was covalently coupled to the N-terminal end of the peptides via the γ-carboxyl of its glutamic acid (Nagy et al., Proc. Nat. Acad. Sci. USA 90:6373, 1993). The cytotoxicity results with HT-1080 tumor cells (a human fibrosarcoma line) are presented in FIG. 10 as a plot of % cell survival against equivalent drug concentration. Cytotoxicity was assayed as described above in Example 4. The cytotoxicity of methotrexate was still reduced when attached to the short peptides, but to a lesser degree that with doxorubicin (see FIGS. 6A-F). As shown in FIGS. 11A-C, methotrexate-PVG was found to be cytotoxic when tested on three human tumor cell lines, namely HT-1080 (a human fibrosarcoma line, FIG. 11A), BT-20 (a human breast carcinoma line, FIG. 11B) and RT-112 (human urinary bladder transitional carcinoma line, FIG. 11C), albeit with a certain degree of neutralization as compared to free methotrexate. Example 8 describes the synthesis of certain dextran-oligopeptide-methotrexate conjugates.

### Example 8

### Synthesis of dextran-oligopeptide-methotrexate Conjugates

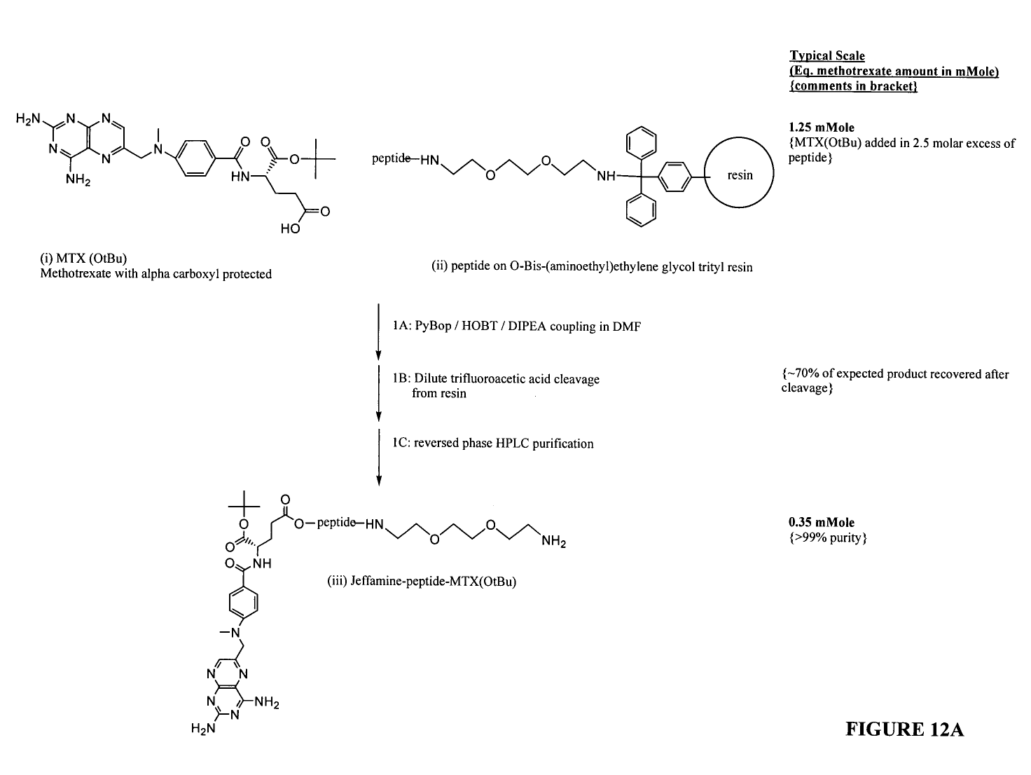

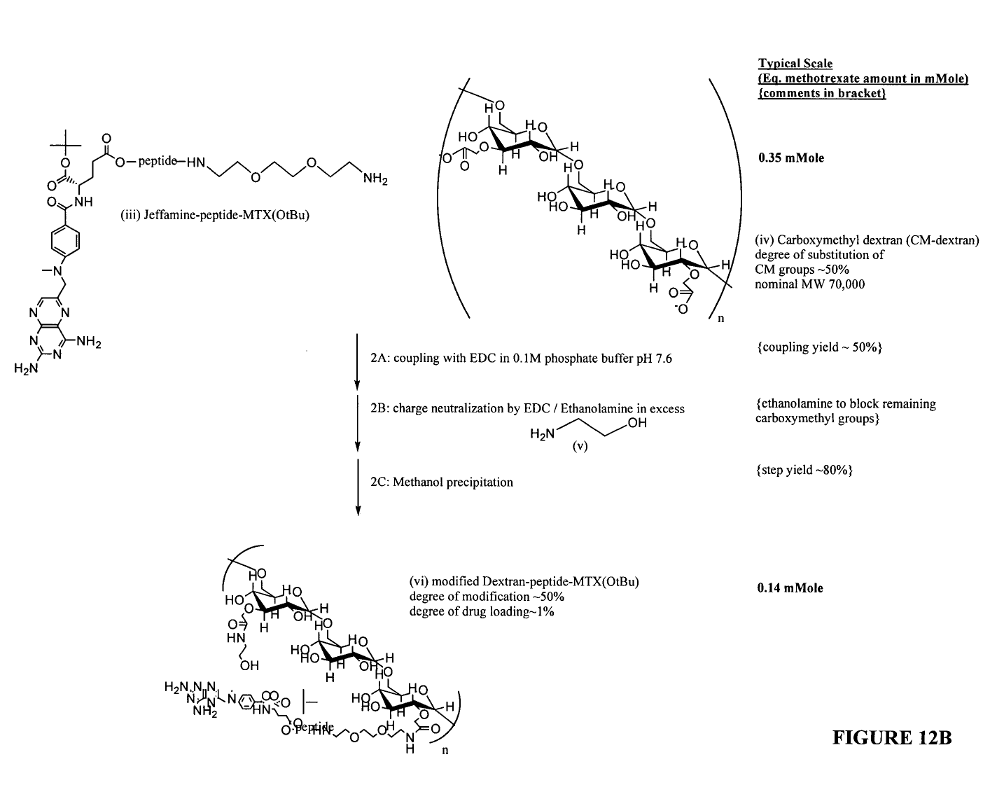

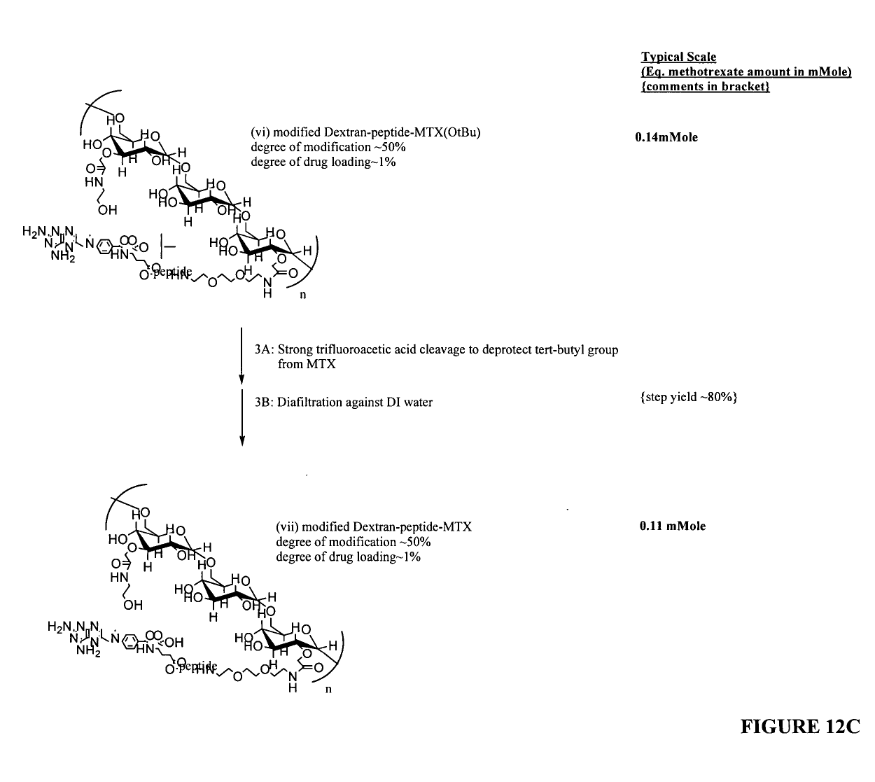

Dextran-oligopeptide-methotrexate conjugates were synthesized following the route of FIGS. 12A-C. The scale of synthesis and the average step yield are also shown. The synthesis route may be modified to prepare conjugates with different oligopeptide recognition segments and different backbone charges. By tuning these parameters, the new conjugate may be optimized in terms of sensitivity against a digestive enzyme of interest and stability in circulation.

1. Materials and Methods

a. Materials

Dextran T-70 (nominal MW of 70,000 Da) was obtained from Pharmacia. 1-ethyl-3-(3-dimethylaminopropyl)carbodiimide hydrochloride (EDC) and ethanolamine were obtained from Alfa Aesar. O-Bis-(aminoethyl)ethylene glycol trityl resin, PyBop and HOBT were obtained from Nova Biochem. Glutamic acid-α-OtBu was purchased from Bachem. Trifluoroacetic acid (TFA), dimethylformamide (DMF), dimethylsulfoxide (DMSO) and acetonitrile (ACN) were obtained from EM Science. Chloroacetic acid, N,N′-diisopropyl(ethylamine) (DIPEA), BOP, 4-amino-4-deoxy-N¹⁰-methylpteroic acid (APA) and other chemicals were from Sigma.

Purified active human MMP-2 was purchased from Calbiochem. Precast protein gels with 10% gelatin were obtained from Biorad. All cell culture media and reagents were ordered from Gibco. Human tumor cell lines HT-1080, U-87 and BT-20 were obtained from American Type Cell Culture. Human bladder tumor cell line RT-112 was a gift from the laboratory of Professor Marsha Moses of the Children's Hospital (Boston, Mass.).

b. Reversed Phase Analytical Assay for Monitoring Process Yield and Product Purity

A Vydac silica C18 0.21 mm ID×25 cm L column was connected to an Agilent 1100 series HPLC solvent delivery system equipped with a UV detector. The method ran at 0.5 ml/min with mobile phase A as 0.2% TFA in water and mobile phase B as 0.2% TFA in acetonitrile. A sample of 10 μL was injected onto a pre-equilibrated column followed by 5 minutes of wash with A. A gradient ramped from 0-100% B in 20 minutes. Detection for methotrexate-containing moieties was made at A307. A calibration curve was constructed correlating the peak areas in chromatograms and concentrations of methotrexate standard.

c. Two Column Size-Exclusion Assay for Enzyme Kinetics Study

A Tosohaas PW3000 7.8 mm ID×30 cm L column followed by a PW2000 7.5 mm ID×30 cm L column were connected to an Agilent 1100 series HPLC solvent delivery system equipped with a UV detector. Isocratic elution was performed with 20% acetonitrile and 3.6× phosphate buffered saline in water at 0.8 ml/min. Detection for methotrexate-containing moieties was made at A307. The default sample injection volume was set at 10 μL. A calibration curve was constructed correlating the peak areas in chromatograms and concentrations of methotrexate standard.

d. One Column Size-Exclusion Assay for Monitoring Process Yield and Product Purity

A Tosohaas PW3000 7.8 mm ID×30 cm L column was connected to an Agilent 1100 series HPLC solvent delivery system equipped with a UV detector. Isocratic elution was performed with 20% acetonitrile and 3.6× phosphate buffered saline in water at 0.8 ml/min. Detection for methotrexate-containing moieties was made at A307. The default sample injection volume was set at 100 μL. A calibration curve was constructed correlating the peak areas in chromatograms and concentrations of methotrexate standard.

e. Zeta Potential Measurement

Dextran, dextran-methotrexate conjugates and dextran-oligopeptide-methotrexate conjugates were dissolved in DI water at 10 mg/ml. Measurement was performed with a Zeta-analyzer from Brookhaven Instruments Corp.

f. Measurement of Kinetic Parameters for MMP-2 Digestion of Conjugates

Conjugates of interest were dissolved in assay buffer (10 mM CaCl₂, 0.2 M NaCl in 50 mM Tris pH 7.5) at a range of concentrations (5, 10, 20, 50 and 100 μM). For each substrate concentration, active MMP-2 at a final concentration of 9.7 nM was added and the mixture was incubated at 37 C. At specific time points, the reaction mixture was quenched with EDTA solution to a final concentration of 20 mM. The mixture was assayed by two-column size exclusion HPLC and the cleavage product peak was quantified to determine the reaction rate. The initial rate was based on the time taken to cleave the first 20% of the conjugate. Triplicate measurements were made at each time point for each substrate concentration. Linear regression was performed on a double reciprocal plot of 1/Vᵢ versus 1/Sₒ to determine the specificity constant k_{cat}/Kₘ according to the Michaelis-Menten equation.

2. Synthesis

a Preparation of Carboxymethyl Dextran (CM-Dextran) [Structure iv, FIG. 12]

The procedure used was based on the method by Nogusa for activating pullulan (Nogusa et al., Chemical & Pharmaceutical Bulletin 43:1931, 1995). 10 g of dextran T-70 was dissolved in 100 ml of 6 N sodium hydroxide solution. 30.2 g of chloroacetic acid was added to the solution and held at 70 C for 2-3 hours. Elevated temperature was necessary for this reaction. The reaction mixture was removed from heat and checked for pH. Acetic acid was used to neutralize the pH if necessary. The solution was then dialyzed against DI water using a Spectrapor regenerated cellulose membrane with a molecular weight cutoff at 3000-8000 for 2 days at 4 C and lyophilized for storage. The degree of carboxymethyl substitution was determined by analysis of sodium content. Atomic absorption analysis was performed at Quantitative Technologies Inc. (Whitehouse, N.J.). The degree of substitution was consistently at 50% (+/−2%).

b. Synthesis and Purification of Methotrexate-alpha-OtBu [Structure i, FIG. 12]

MTX-α-(OtBu) was prepared from 4-amino-4-deoxy-N¹⁰-methylpteroic acid (APA) and glutamic acid-α-OtBu using BOP as the conjugating reagent, following the procedure of Nagy (Nagy et al., Proc. Natl. Acad. Sci. USA 90:6373, 1993). Crude MTX-α-(OtBu) was precipitated by centrifugation in a 1:1 mixture of cold ether/ethyl acetate. The material was further purified by HPLC on a Vydac C18 preparative scale column using a gradient from 0-100% acetonitrile with 0.2% TFA. The organic solvent in product fractions was removed with RotaVap and the purified material was subsequently lyophilized. The product purity was confirmed to be ≧95% and the amount was quantified by analytical scale reversed-phase HPLC. The average retention factor was 6.14.

c. Synthesis of Jeffamine-oligopeptide-methotrexate(OtBu) [Structure iii, FIG. 12]

Standard FMOC peptide synthesis in solid phase was carried out on O-Bis-(aminoethyl)ethylene glycol trityl resin by the MIT Biopolymer Lab (Cambridge, Mass.). As in conventional peptide synthesis, the peptide chain grew from carboxyl to amino terminals. After the last amino acid was loaded to the chain, FMOC was removed and the oligopeptide with a free amino terminal was exposed. The carboxyl terminus remained attached to the solid resin via a 6 carbon polyethylene oxide spacer.

To couple MTX-α-OtBu to the amino terminal of the oligopeptide chain, MTX-α-OtBu (2.5 eq.) was dissolved in DMF with DIPEA (2.5 eq.). PyBop (2.5 eq.), HOBT (2.5 eq.) and another 2.5 eq. of DIPEA were added to activate the γ-carboxyl of MTX-α-OtBu for about 15 minutes. More DMF was added such that the final concentration of MTX-U-OtBu was about 0.1 M. The mixture was added to the resins loaded with oligopeptide and stirred at room temperature overnight. The reaction was confirmed for completeness the next day using the ninhydrin test. Excessive reactants were washed away from resins with DMF, DCM and methanol. Resins were dried thoroughly.

Jeffamine-oligopeptide-MTX(OtBu) was cleaved from the trityl resin by suspending the resins in 2% TFA in DCM for 10 minutes. This step was repeated 3 times. To collect all the cleaved product, the resins were further washed with DCM and methanol and the step was repeated until no further yellow color was observed (the yellow color is indicative of the presence of the MTX(OtBu) containing product). Organic solvent was then removed from the collected product by RotaVap. The crude material was resuspended in water and purified by HPLC over a 0-100% acetonitrile gradient with 0.2% TFA using a 95 ml Vydac C18 preparative column. Product fractions were lyophilized after organic solvent was removed by RotaVap. Product purity was found to be ≧95% and the amount was quantified by analytical scale reversed phase HPLC. The average retention factor was 6.71. Mass spectroscopy confirmed the identity of product (with Pro-Val-Gly-Leu-Ile-Gly as oligopeptide in jeffamine-oligopeptide-MTX(OtBu), calculated mass: 1177.3; observed mass: 1177.7).

d. Synthesis of Jeffamine-methotrexate(OtBu) [Structure iii, Not Shown]

The procedure was similar to that described above in (c) except that no oligopeptide was loaded onto the resin. MTX-α-OtBu was directly coupled to O-Bis-(aminoethyl)ethylene glycol trityl resin using the same protocol. The product was found to be ≧95% by reversed-phase HPLC. The average retention factor was 5.81. Mass spectroscopy confirmed product identity (calculated mass: 640.6; measured mass: 640.6).

e. Synthesis and Purification of Dextran-oligopeptide-MTX[Structure vii, FIG. 12]

To couple jeffamine-oligopeptide-MTX(OtBu) to CM-dextran, 0.5 mmole of jeffamine-oligopeptide-MTX(OtBu) [structure iii] was dissolved in 1.9 ml DI water and then added to 4 g of CM-dextran [structure iv] dissolved in 6.7 ml of DI water. 4.7 g of EDC was added to the mixture with stirring. Reaction was held overnight at room temperature. Another 4.7 g of EDC was added the next day and the reaction continued overnight to form CM-dextran-oligopeptide-MTX(OtBu).

To block unreacted carboxymethyl groups on the dextran carrier, 30 ml of ethanolamine was first neutralized to pH 6-7 using 5 N HCL in dibasic phosphate buffer on ice. The mixture was added to the CM-dextran-oligopeptide-MTX(OtBu) followed by 47 g of EDC. The reaction mixture was stirred at room temperature overnight. Dextran-oligopeptide-MTX(OtBu) after neutralization was precipitated by centrifugation in methanol.

To remove the protection group OtBu, modified dextran-oligopeptide-MTX(OtBu) was resuspended in 40 ml strong trifluroacetic acid by vigorous stirring and held at room temperature for 3 hours. The deprotection step was quenched on ice by diluting in 0.1 M phosphate buffer and neutralizing the pH with 5 N sodium hydroxide. Extensive diafiltration was performed using Millipore Pellicon XL diafiltration cassettes of regenerated cellulose with 10,000 MWCO to remove excessive reactants and byproducts of reaction. After purification, the product in the retentate was concentrated and lyophilized for storage. The product purity was confirmed to be ≧99% and the loading density was quantified by size exclusion HPLC. The loading density was estimated to be at 1+/−0.2% (mole % per unit glucose unit).

f. Synthesis and Purification of Dextran-MTX[Structure vii' Not Shown]

The procedure was similar to that described above in (e) except that jeffamine-MTX(OtBu) was used instead of jeffamine-oligopeptide-MTX(OtBu).

3. Discussion

a. Synthesis of Oligopeptide-Drug Product

As described in Example 7, methotrexate was selected to form the drug component of these inventive conjugates because it exhibits bulk tolerance at the γ-carboxyl group. The less tolerant α-carboxyl group was protected throughout the synthesis process by preparing MTX-α-(OtBu) [structure i] from APA. The α-protected methotrexate was conjugated to the recognition oligopeptide in a solid phase reaction. Trityl resin was chosen as the solid phase support so that the oligopeptide-drug product [structure iii] could be released from the resin using dilute acid that did not affect the tert-butyl protecting group on methotrexate. The tert-butyl group was removed in the last reaction step using strong TFA cleavage (step 3A, FIG. 12).

b. Addition of a Spacer Decreases Steric Hindrance Between the Recognition Segment and Polymeric Carrier

Experience with the conjugates of Example 1 indicated that lack of spacing between the oligopeptide recognition sequence and the polymer carrier may reduce the rate of enzymatic cleavage as a consequence of steric hindrance. The recognition segment and polymeric carrier of the methotrexate conjugates were therefore separated by a ten atom hydrophilic and flexible spacer called Jeffamine (2,2′-(ethylenedioxy)diethylamine). A special trityl resin, O-Bis-(aminoethyl)ethylene glycol trityl resin [structure ii] was used as the solid support thereby enabling the recognition segment to be directly attached to Jeffamine on the solid phase, thus simplifying the purification task.

Jeffamine also aided in coupling the polymeric carrier [structure iv] and the oligopeptide-drug product [structure iii]. Indeed, the selected oligopeptide sequence for MMP-2 recognition includes hydrophobic amino acids (Pro-Val-Gly-Leu-Ile-Gly) and the protected drug MTX-α-(OtBu) has limited water solubility. On the other hand, the polymeric carrier is composed of hydrophilic CM-dextran. Attempts to couple the oligopeptide-drug and polymeric carrier in the presence of EDC, revealed that the concentration of amine components [structure iii] and carboxyl components [structure iv] affected conjugation yield. Table 4 lists the conjugation yields that were obtained when the amine and/or carboxyl concentrations were varied (all other reaction conditions were unchanged).

TABLE 4

| Amine | Carboxyl | Conjugation |
|---|---|---|
| concentration (M) | concentration (M) | Yield (%) |
| 0.02 | 0.66 | 76 |
| 0.02 | 0.18 | 41 |
| 0.02 | 0.09 | 29 |
| 0.02 | 0.02 | 15 |
| 0.01 | 0.66 | 25 |

These observations are thought to be caused by the relatively short half-life of the EDC-activated carboxyl. Indeed, when EDC is unable to encounter an amine component for reaction, the active ester becomes inactivated (Hermanson, Bioconjugate Techniques, Academic Press, 1996). The hydrophilic character of the jeffamine spacer may increase the water solubility of the oligopeptide-drug product [structure iii] thereby enabling conjugation in aqueous phase to achieve reasonable yield (step 2A, FIG. 12).

c. Varying the Backbone Negative Charge of the Polymeric Carrier

Experience with the conjugates of Example 1 indicated that the rate of MMP-2 cleavage tends to increase as the ionic strength of the digestion medium is increased (see Table 3, Example 2). Debye-Hückel theory would suggest that this is caused by electrostatic repulsion between the enzyme and conjugate because of the negative charge on the CM-dextran backbone (Chau and Langer, Second International Symposium on Tumor Targeted Delivery Systems in Rockville, Md., “Important factors in designing targeted delivery of cancer therapeutics via MMP-2 mediation”, 2002). Unreacted carboxymethyl groups were therefore blocked with excess ethanolamine [structure v], a neutral and hydrophilic molecule (step 2B, FIG. 12). The charge on the polymer backbone was determined by measuring the Zeta potential. As shown in Table 5, tests with CM-dextran [structure iv] showed that the negative charge could be fully neutralized under appropriate reaction conditions.

TABLE 5

| Sample | Zeta potential (mV) |
|---|---|
| Dextran T70¹ | −6.60 |
| CM-Dextran² | −47.40 |
| Ethanolamine neutralized CM-Dextran A³ | −36.25 |
| Ethanolamine neutralized CM-Dextran B⁴ | −1.06 |

¹Neutral.
²Degree of substitution ˜50% determined by atomic absorption sodium analysis.
³EDC at 2 fold excess and ethanolamine at 10 fold excess of glucose units present on dextran.
⁴EDC at 10 fold excess and ethanolamine at 20 fold excess of glucose units present on dextran.

By tuning the amount of EDC and ethanolamine, two dextran-PVGLIG-methotrexate conjugates with different negative charges were prepared. The kinetics of MMP-2 digestion were also assessed as shown in Table 6.

TABLE 6

| Sample | Zeta potential (mV) | k_{cat}/Kₘ (M⁻¹ · s⁻¹) |
|---|---|---|
| Conjugate C1 | −27.8 | 1.09 × 10⁴ |
| Conjugate C2 | −12.2 | 1.22 × 10⁵ |

The sensitivity constant of the less negative conjugate C2 was comparable to the value of 0.82×10⁵ that was measured by Turk with the optimal oligopeptide Ile-Pro-Val-Ser-Leu-Arg-Ser (Turk et al., Nature Biotechnology 19:661, 2001). Interestingly, none of the peptide motifs of the MMP-2 recognition sequence found in the literature carry a negatively charged amino acid (Turk et al., Nature Biotechnology 19:661, 2001; Netzel-Amett et al., Analytical Biochemistry 195:86, 1991; Knight et al., FEBS Letters 296:263, 1992; Xia et al., Bioch. Biophys. Acta 1293:259, 1996). These reports are in agreement with the observation that the conjugate with the least negative backbone charge demonstrates the fastest digestion with MMP-2.

### Example 9

### Peptidyl Methotrexate Release in the Presence of MMP-2

Dextran-PVGLIG-methotrexate and dextran-methotrexate conjugates from Example 8 were incubated with 14.7 nM active human MMP-2 in MMP-2 assay buffer at 37 C for 6 hours and assayed for percentage cleavage using size exclusion chromatography. Dextran-PVGLIG-methotrexate conjugate released more than 90% of methotrexate whereas dextran-methotrexate did not show any significant release. The comparison confirms that the labile oligopeptide segment promotes cleavage of the conjugate. Without the recognition segment, the conjugate was stable even in the presence of a high concentration of MMP-2. The small molecular weight cleavage product was collected from the digest of dextran-PVGLIG-methotrexate by size exclusion chromatography and analyzed by mass spectroscopy. The molecular weight of 707.5 was consistent with the notion that methotrexate-Pro-Val-Gly (MTX-PVG) was the released product after cleavage.

### Example 10

### Stability of Dextran-oligopeptide-methotrexate Conjugates in Serum

As noted previously it is preferable that the conjugates remain stable in blood circulation and that the drug stays attached to the polymer backbone before reaching the target tissue. When tumor tissues express a high level of MMP-2, there will be an elevated enzyme level in the blood stream (Kuyvenhoven et al., Thrombosis and Haemostasis 89:718, 2003). However, protease activity is inhibited by serum proteins such as α-2-macroglobulin (Woessner and Nagase, Matrix Metalloproteinases and TIMPs, Oxford University Press, 2000). The stability of dextran-PVGLIG-methotrexate conjugates of Example 8 were therefore measured in both fetal bovine serum and fetal bovine serum spiked with active human MMP-2 (at 10 nM final concentration). After incubating the conjugates at 37 C in these conditions for 24 hours, no significant cleavage was detected as assayed by size exclusion chromatography.

### Example 11

### In Vitro Cytotoxicity of Peptidyl Methotrexate

As discussed in Example 7, the cytotoxicity of peptidyl methotrexate and free methotrexate were found to be comparable when tested on three human tumor cell lines (see also FIGS. 1A-C).

### Example 12

### In Vivo Evaluation of Anti-Tumor Efficacy of Dextran-oligopeptide-methotrexate Conjugates

The following example describes experiments that were performed to test the in vivo efficacy of the dextran-oligopeptide-methotrexate conjugates of Example 8. Six week old female SCID mice (obtained from Charles River Laboratory) were subcutaneously injected with 10⁶ HT-1080 tumor cells (obtained from ATCC) at mid-dorsal. HT-1080 is a human fibrosacroma cell line that is known to express high level of MMP-2. After 7-10 days, tumors of size 150-300 mm³ were established on each mouse. Free methotrexate, dextran-oligopeptide-methotrexate (with MMP-2 labile oligopeptide segment Pro-Val-Gly-Leu-Ile-Gly) or dextran-methotrexate were injected intraperitoneally on day 1, 8 and 15 after a tumor was first established. Weight and tumor size were monitored three times a week. Tumor size was calculated as width²×length×0.52. To ensure that the modified dextran carrier of the conjugates did not induce toxicity, a first control group of mice were injected with dextran that was charge neutralized with ethanolamine. As a second control, another group of mice were injected with phosphate buffered saline. Each group included three mice.

The dosage chosen for these experiments was based on the reported optimum dosage of free methotrexate for treating tumor-bearing mice (Burger et al., Int. J. Cancer 92:718, 2001). Three weekly injections at 50 mg of methotrexate/kg of body weight was half the maximum tolerated dosage. In another set of experiments the dosage was increased to three weekly injections of 100 mg/kg, or to injections of 50 mg/kg twice per week. These higher dosages resulted in serious toxic or lethal responses and body weight dropped more than 20% (data not shown).

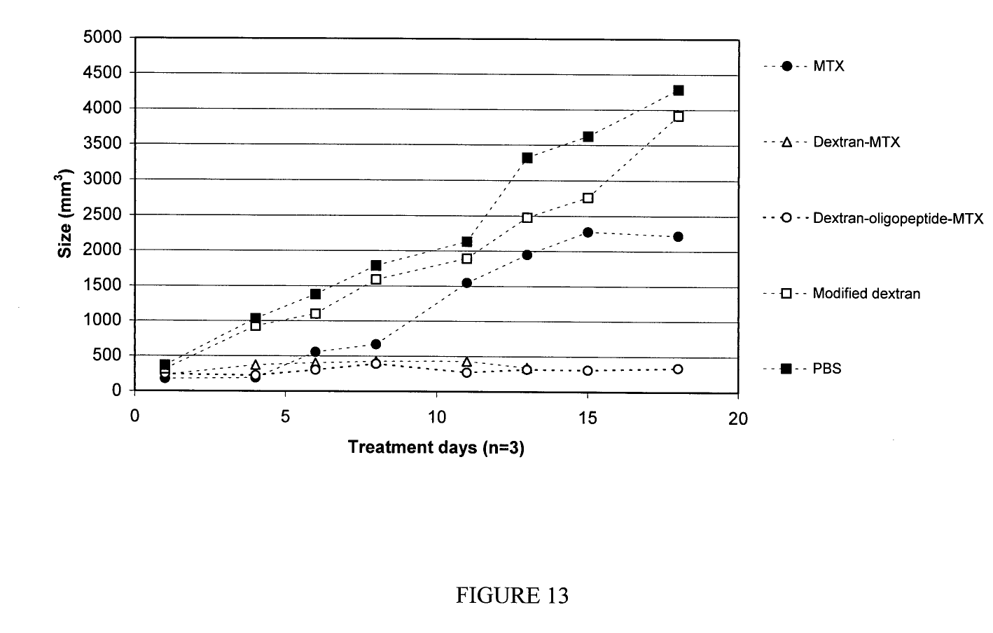

FIG. 13 shows the tumor size progression after treatment, as an indication of treatment efficacy. Free methotrexate suppressed tumor growth by 48% at the end of the study compared to saline control. Dextran-methotrexate and dextran-oligopeptide-methotrexate conjugates were both more effective in hampering the progression of tumor. At the end of study, average tumor size in mice treated by either conjugate was suppressed by 92%.

Linking methotrexate to dextran increases the half-life of the small molecule drug due to decreased renal elimination, rendering the benefit of passive targeting. As described above, the leakiness of tumor blood vessels as compared to other normal tissue further increases the targeting ratio. As an anti-metabolite, methotrexate must be released from the carrier to exert its effect on cell growth by inhibiting DNA synthesis. The release can happen in two ways, namely (1) by non-specific endocytosis, whereby conjugates are internalized by cells and methotrexate can be released by either acid hydrolysis or lysosomal enzyme digestion; and (2) by MMP-2 cleavage of the oligopeptide segment in the extracellular space of the tumor tissue. The latter route is only possible for conjugates with an MMP-2 labile oligopeptide segment between methotrexate and the dextran carrier.

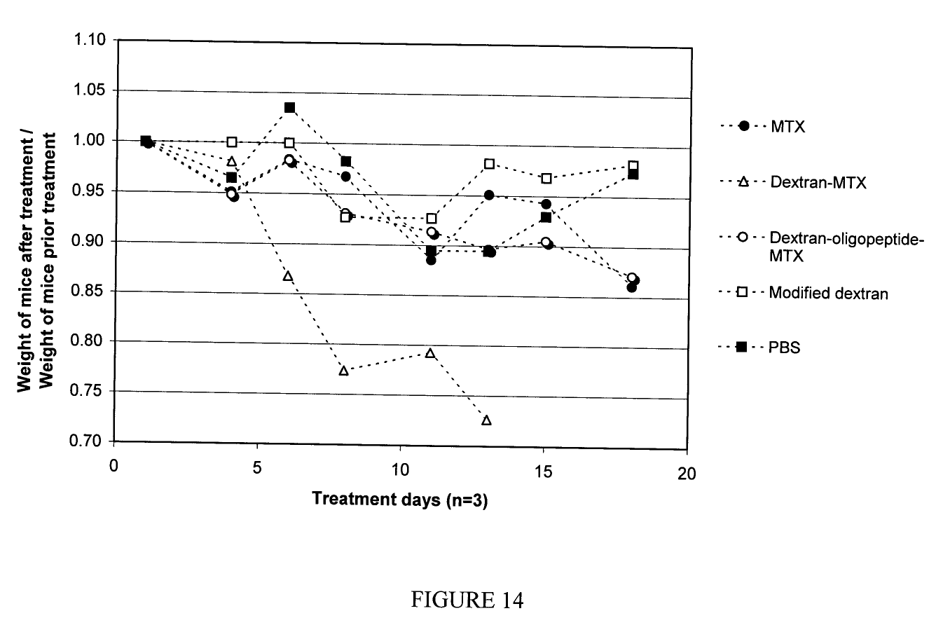

The body weight of the mice was also monitored as an indicator of toxic side effects (see FIG. 14). The polymeric carrier, modified dextran, showed no toxicity. At equivalent dosage, methotrexate and dextran-oligopeptide-methotrexate caused similar percentage weight drops (13% and 14% respectively compared to initial weight). The significantly better efficacy of dextran-oligopeptide-methotrexate here demonstrates the advantage of targeting. The enhanced anti-tumor effect of dextran-oligopeptide-methotrexate is presumably a result of altered biodistribution of methotrexate, with more drug delivered to the targeted tumor tissue. Free methotrexate is a drug of low molecular weight (MW=454.45 Da) and has a short plasma half-life. As a result, the total body clearance is high and only small amount of the injected dose reaches the tumor tissue. The dextran-methotrexate and dextran-peptide-methotrexate conjugates behave differently. Because the dextran carrier is larger than the renal excretion limit, the plasma half-life is longer and this enables a larger fraction of the injected dose to reach the tumor tissue. This advantage is termed passive targeting.

Dextran-methotrexate, albeit showing promising efficacy, was significantly more toxic than dextran-oligopeptide-methotrexate, causing a 27% drop in body weight. In fact, the study for this group was terminated at an earlier stage because of severe toxicity (Day 13, see FIGS. 13 and 14). It is speculated that the MMP-2 labile linker is responsible for the lower toxicity of the dextran-oligopeptide-methotrexate. Indeed, in normal tissue without MMP-2, free methotrexate from dextran-methotrexate conjugate is potentially released as a result of non-specific endocytosis. In contrast, peptidyl-methotrexate is released from dextran-oligopeptide-methotrexate conjugates. Because of the lower cytotoxicity of the peptidyl-methotrexate versus the free drug, a lower toxic response is seen in normal tissues.

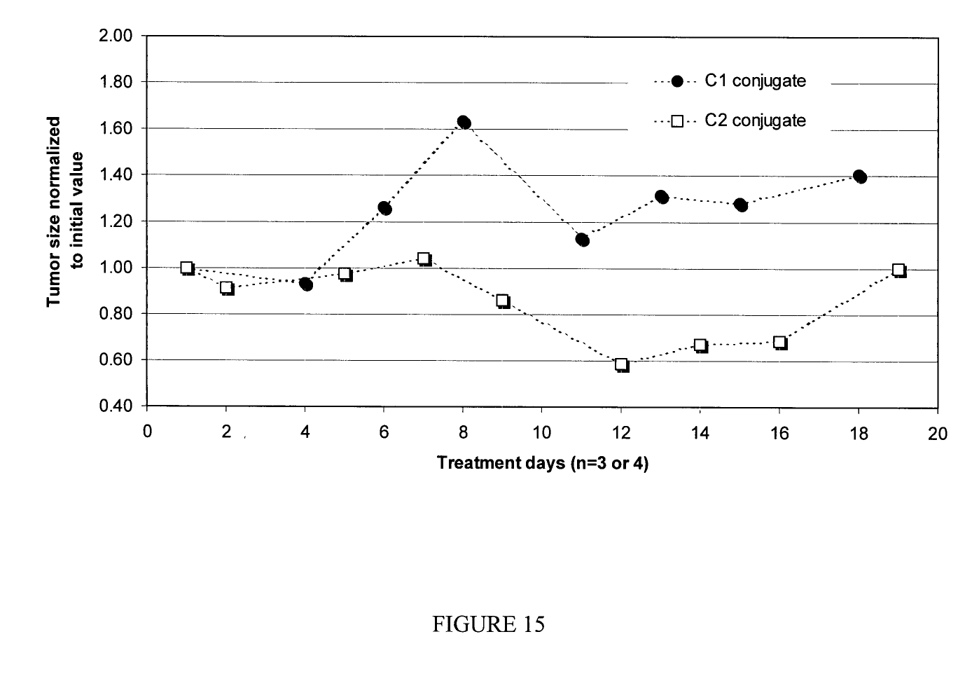

If the tumor associated enzyme MMP-2 mediates targeted drug release for the new dextran-oligopeptide-methotrexate conjugate, one would expect a conjugate that is more sensitive to MMP-2 in vitro to generate a more pronounced therapeutic effect. The in vivo efficacy of the two dextran-oligopeptide-methotrexate conjugates of Table 6, Example 8 was therefore compared as illustrated in FIG. 15. Conjugate C2 that is more sensitive to MMP-2 was more effective in suppressing tumor growth. Tumor size was approximately the same as the initially established size by the end of the study. In comparison, a tumor size increase of 40% was observed when the mice were treated with the less sensitive conjugate C1. In addition conjugate C2 was slightly less toxic than C1. Mice treated with conjugate C2 lost 8% body weight while mice treated with C1 lost 13% by the end of study (data not shown).

### OTHER EMBODIMENTS

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A conjugate for use in targeting a drug to a tissue, wherein a digestive enzyme is overexpressed in the extracellular space of the tissue, the conjugate comprising:
a polymeric carrier;
a drug molecule; and
a linker that includes a first end and a second end, wherein the polymeric carrier is associated with the first end of the linker and the drug is associated with the second end of the linker and wherein the linker includes a recognition segment that is cleaved when the conjugate is exposed to the digestive enzyme.

2. The conjugate of claim 1 further comprising:
additional drug molecules; and
additional linkers, wherein each drug molecule is indirectly associated with the polymeric carrier via one of the linkers and wherein each linker includes a recognition segment that is cleaved when the conjugate is exposed to the digestive enzyme.

3. The conjugate of claim 1, wherein the polymeric carrier is hydrophilic, biocompatible and biodegradable.

4. The conjugate of claim 1, wherein the polymeric carrier is larger than the renal excretion limit.

5. The conjugate of claim 1, wherein the drug is a small molecule drug.

6. The conjugate of claim 1, wherein the drug is a biomolecular drug.

7. The conjugate of claim 1, wherein the recognition segment is an oligopeptide.

8. The conjugate of claim 1, wherein the recognition segment is an oligosaccharide.

9. The conjugate of claim 1, wherein the tissue is diseased.

10. The conjugate of claim 9, wherein the tissue is a tumor.

11. A pharmaceutical composition comprising a pharmaceutically acceptable excipient and an effective amount of the conjugate of claim 1.

12. A method of preparing a conjugate for use in targeting a drug to a tissue, wherein the tissue overexpresses a digestive enzyme, the method comprising:
providing a polymer carrier;
providing a drug molecule;
providing a linker that includes at least a first end and a second end, wherein the linker includes a recognition segment that is cleaved when the conjugate is exposed to the digestive enzyme;
associating the polymer carrier with the first end of the linker; and
associating the drug molecule with the second end of the linker.

13. A method of administering a drug to a patient, the method comprising steps of:
providing a patient;
providing a pharmaceutical composition that comprises a pharmaceutically acceptable excipient and an effective amount of the conjugate of claim 1; and
administering the pharmaceutical composition to the patient.
